(19) United States
(12) Patent Application Publication
(10) Pub. No.: US 2011/0110974 A1
(43) Pub. Date: May 12, 2011

(54) METHODS AND KITS FOR INDUCING A CTL RESPONSE USING A PRIME BOOST REGIMEN

(76) Inventors: Erik Depla, Destelbergen (BE); Annegret Van Der AA, Berendrecht (BE)

(21) Appl. No.: 12/734,397

(22) PCT Filed: Oct. 28, 2008
(86) PCT No.: PCT/EP2008/064585

§ 371 Date: Jan. 21, 2011

Related U.S. Application Data
(60) Provisional application No. 61/000,749, filed on Oct. 29, 2007.

(30) Foreign Application Priority Data
Oct. 29, 2007 (EP) 07119507.7

(51) Int. Cl. A61K 39/12; A61K 39/00; A61P 37/00

(52) U.S. Cl. 424/204.1; 424/184.1

(57) ABSTRACT

The present invention relates to the generation of a T cell response against a target antigen using a polypeptide comprising a polyepitope construct as a priming composition in a prime boostregimen.

### FIELD OF THE INVENTION

The present invention relates to the generation of a T cell response against a target antigen using a polypeptide comprising a polyepitope construct as a priming composition in a prime boost regimen.

### BACKGROUND ART

The main bottleneck in developing vaccines for intracellular infections such as HBV, HCV, HIV, malaria and chronic diseases such as cancer is the ability to induce strong and long-lasting cell-mediated immunity. Stimulation of a functional CD8+ response is often crucial in addition to a Th1-type CD4+ T cell response. DNA immunization was originally shown to induce strong cytotoxic T lymphocyte (CTL) responses in murine models but it is now clear that induction of cell-mediated immunity by DNA vectors is not as potent in humans and thus new adjuvants and antigen (Ag) delivery systems are being developed for improved immunogenicity. The use of recombinant viral vectors is an increasingly popular alternative to achieve intracellular Ag expression that can result in antigen presentation on MHC class I molecules thus allowing the induction of CD8+ T cell responses.

Based on data shown in literature (McConkey et al 2003, Mwau et al. 2004, Vuola et al 2005) heterologous prime-boost vaccination regimens that combines two different vectors encoding the same antigen is more efficient in inducing cell mediated immune response than the use of a single vector.

In view of the heterogeneous immune response observed with viral infection, induction of a multi-specific cellular immune response directed simultaneously against multiple epitopes is important for the development of an efficacious vaccine.

The technology relevant to polyepitope vaccines is developing and a number of different approaches are available which allow simultaneous delivery of multiple epitopes.

Vaccines currently under development and comprising multiple CTL and/or HTL epitopes are based on genetic constructs delivered either by viral vectors or naked DNA immunizations. Subunit vaccines based on one or more purified proteins require effective adjuvant systems to induce a strong immune response. More specific, vaccines based on recombinant or purified proteins are generally effective in inducing T helper lymphocytes (HTL) and antibody responses, but are generally ineffective at induction of CTL responses (Alexander et al., 2002). This limitation presents a serious drawback for vaccines and immunotherapeutic regimens targeting diseases for which induction of CTL responses appears to be important.

Polypeptides have been synthesized or produced by recombinant expression technologies, but as mentioned, their use as vaccines has resulted in generally only antibody and HTL responses. The use of CTL inducing adjuvants (e.g. Freunds Adjuvant, AS02, ISCOMATRIX™-adjuvant) or specific “particle formation promoting proteins” or “carrier proteins” (e.g. Ty-VLP, CyaA, Neisseria P64K) are generally required for higher levels of immunogenicity and induction of CTL responses.

Moreover, large polypeptides (more than 50 amino acids) are extremely difficult to synthesize and purify at large scale. In addition, non-native polypeptides comprising multiple epitopes have been proven to be difficult to produce in bacterial or mammalian expression systems due to high levels of degradation (Thomson et al. 1996).

As such, heterologous prime-boost applications have mainly focussed on the use of combinations including DNA-viral vector or two types of viral vectors. Limitations of these systems include lack of potency of DNA in man and the pre-existing immunity induced against viral vectors which restricts the number of immunizations.

It has now been found that the drawbacks in the prior art can be overcome by using a protein in a prime boost regimen. Remarkably, the use of a polypeptide comprising a polyepitope construct as a priming composition induces a very high CTL response against a target antigen when applied in a heterologous prime boost regimen and does not require the need for CTL inducing adjuvants, particle formation promoting proteins or carrier proteins.

### SUMMARY OF THE INVENTION

The present invention is directed to the use of a polypeptide, comprising a polyepitope construct, as a priming agent in a heterologous prime boost regimen. The invention encompasses a polypeptide for use in inducing a T cell response against at least one target antigen in a prime boost treatment regimen.

In a first embodiment, the invention envisages the use of a polypeptide comprising a polyepitope construct comprising at least two CTL epitopes for the manufacture of a medicament for priming a T cell response against at least one target antigen in a prime boost treatment regimen. More particular, the polypeptide is not linked to, combined with or embedded within a compound selected from the group consisting of: a particle formation promoting protein, a carrier protein and a CTL response inducing adjuvant. Optionally, the polypeptide is formulated in alum. More specific, the T cell response comprises a Cytotoxic T Lymphocyte (CTL) response and optionally a T Helper (HTL) response.

In a specific embodiment, the polyepitope construct comprises at least 10, 11, 12, 13, 14, 15, 16, 17, 18, 18, 20, 25, 30, or up till 150 CTL epitopes. Preferably, the epitopes are isolated CTL epitopes. Optionally, the polyepitope construct further comprises at least 1, 2, 3, 4, 5, 10, 15 or up to 50 HTL epitopes.

In a further embodiment, two or more of the CTL and/or HTL epitopes in the construct are linked by one or more spacer amino acids.

In a further embodiment, the T cell response is directed against at least one target antigen. Preferably, the target antigen is derived from a virus. More preferably, the target antigen is derived from HBV or HCV.

In specific embodiment, the T cell response is directed against the Hepatitis B virus. More particular, the two or more CTL epitopes of the polyepitope construct are derived from the HBV Core protein, the HBV polymerase protein and/or the HBV Envelope protein. More particular, the two or more CTL epitopes of the polyepitope construct are selected from the group of epitopes as given in Table 1. Even more particular, the polyepitope construct further comprises at least one HTL epitope selected from the group of epitopes as given in Table 2.

In an alternative embodiment, the T cell response is directed against the Hepatitis C virus. More particular, the two or more CTL epitopes of the polyepitope construct are derived from the HCV CORE, E1, E2, NS3, NS4 and/or NS5 protein.

More particular, the two or more CTL epitopes of the polyepitope construct are selected from the group of epitopes as given in Table 3. Even more particular, the polyepitope construct further comprises at least one HTL epitope selected from the group of epitopes as given in Table 4.

In a specific embodiment, the polypeptide comprising the polyepitope construct of the present invention is the result of a bacterial or yeast expression. More specific, the polyepitope construct is a recombinant string of two or more CTL epitopes.

Another aspect of the invention relates to a heterologous prime boost treatment regimen, wherein the polypeptide as described herein is used as a priming agent. In a specific embodiment, the invention envisages the use of a polypeptide comprising a polyepitope construct comprising at least two CTL epitopes for the manufacture of a medicament for priming a T cell response against at least one target antigen in a prime boost treatment regimen, wherein the polypeptide is not linked to, combined with or included within a compound selected from the group consisting of: a particle formation promoting protein, a carrier protein and a CTL response inducing adjuvant, and wherein the prime boost treatment regimen comprises the steps of:
a. administering the polypeptide as a priming agent; and
b. subsequently administering a boosting agent comprising a vector encoding one or more CTL epitopes of the target antigen, including at least one CTL epitope which is the same as a CTL epitope of the priming agent.

More particular, the T cell response comprises a Cytotoxic T Lymphocyte (CTL) response and optionally a T Helper (HTL) response.

In a particular embodiment, the medicament is prepared for the administration in a prime boost regimen as described herein.

In a specific embodiment, the vector for use as a boosting agent is a plasmid, a viral vector, a bacterial vector or a yeast vector. More specific, the viral vector is a poxvirus vector. Even more specific, the viral vector is a vaccinia virus vector. In a further embodiment, the vector for use as a boosting agent is a non-replicating or replication impaired viral vector. Preferably, the viral vector is MVA.

In a further embodiment, the vector for use as a boosting agent further encodes for one or more HTL epitopes. Specifically, at least one HTL epitope is a PADRE® epitope. More specifically, the epitopes of the boosting agent are the same as the epitopes of the priming agent, and this in the same or a different order.

It is a further embodiment of the invention that the priming and/or boosting agent is in particulate form suitable for intravenous, intraepidermal, subcutaneous, intradermal, transdermal, or intramuscular delivery.

A further aspect of the invention envisages a specific prime boost treatment regimen using the polypeptide of the present invention as a priming agent.

More specific, the prime boost treatment regimen comprises a priming step with 2 or 3 polypeptide administrations followed by a boosting step with a vector by one, optionally two, administrations. Preferably, the priming and boosting step are separated by at least two weeks.

It is generally noted that the wording “use of the polypeptide in the manufacture of a medicament for priming a T cell response . . . ” in the present invention can alternatively be construed as “the polypeptide for use in priming a T cell response . . . ”.

Another aspect of the invention relates to a method and kit for the use of the present invention. Specifically, the method and kit induce a T cell response against at least one target antigen. More particular, said kit comprises a priming polypeptide as described herein, comprising a polyepitope construct comprising two or more CTL epitopes of the target antigen, wherein the polypeptide is not linked to, combined with or included within a compound selected from the group consisting of: a particle formation promoting protein, a carrier protein and a CTL response inducing adjuvant. More particular, the polyepitope construct comprises at least 15 isolated CTL epitopes. Even more particular, the polyepitope construct further comprises one or more HTL epitopes. Preferably, two or more of the epitopes in the construct are linked by one or more spacer amino acids. In a further embodiment, the kit also comprises a boosting vector, as described herein, encoding one or more CTL epitopes of the target antigen, including at least one CTL epitope which is the same as a CTL epitope of the priming composition. Optionally, the polypeptide is formulated in alum. More specific, the epitopes of the priming composition are the same as the epitopes of the boosting composition.

More particular, the vector of the boosting composition in the kit is a plasmid, a viral vector, a bacterial vector or a yeast vector. Preferably, the viral vector is a poxvirus vector. More preferably, the vector is a vaccinia virus vector. In a further embodiment, the viral vector of the boosting composition in the kit is a non-replicating or replication impaired vector. Even more preferably, the viral vector is MVA.

In a further embodiment of the kit, the target antigen is a virus, and preferably HBV or HCV. More specific, the two or more CTL epitopes are derived from the HBV Core protein, the HBV polymerase protein, the HBV Envelope protein, the HCV Core protein, the HCV E1 protein, the HCV E2 protein, the HCV NS3 protein, the HCV NS4 protein and/or the HCV NS5 protein. In a particular embodiment, the two or more CTL epitopes are selected from the list of epitopes given in Table 1 and/or Table 3. Optionally, at least one HTL epitope is selected from the list given in Table 2 and/or Table 4.

In a further embodiment, the priming and/or boosting composition of the kit is in particulate form suitable for intravenous, intraepidermal, subcutaneous, intradermal, transdermal, or intramuscular delivery.

### FIGURE LEGENDS

FIG. 1: Schematic of the HBV DNA construct INX102-3697. The orientation of the CTL and HTL epitopes in the synthetic gene is shown in the upper part. The polyepitope cassette consist of a signal sequence, a CTL domain (first row: 30 CTL epitopes and PADRE) and an HTL domain (second row: 16 HTL epitopes). The HLA restriction of each epitope, with respect to supertype, is also shown. The functional elements of the DNA plasmid vector are indicated in the lower part of the figure.

FIG. 2: CTL responses obtained after homologous prime-boost immunization with protein compared to heterologous protein prime/MVA boost. Cumulative amount of specific IFN-γ spots in CD8+ spleen cells direct ex vivo, after stimulation with 6 HLA-A2-restricted HBV epitopes, loaded on Jurkat A2.1/K^{b} cells. The number of specific spots (delta versus set-up with unloaded Jurkat A2.1/K^{b} cells) is given by the bars.

FIG. 3: Th1 responses obtained after homologous prime-boost immunization with protein compared to heterologous protein prime/MVA boost. Cumulative amount of specific IFN-γ spots in CD4+ spleen cells direct ex vivo, after stimulation with HLA-DR-restricted HBV epitopes and PADRE, loaded on syngeneic spleen cells. The number of specific spots (delta) is given by the bars.

FIG. 4: Th2 responses obtained after homologous prime-boost immunization with protein compared to heterologous protein prime/MVA boost. Cumulative amount of specific IL-5 spots in CD4+ spleen cells direct ex vivo, after stimulation with HLA-DR-restricted HBV epitopes and PADRE, loaded on syngeneic spleen cells. The number of specific spots (delta) is given by the bars.

FIG. 5: CTL responses obtained after heterologous protein prime/MVA boost using non-adjuvanted or aluminium formulated protein compared to immunization with MVA only. Cumulative amount of specific IFN-γ spots in CD8+ spleen cells direct ex vivo, after stimulation with 6 HLA-A2-restricted HBV epitopes, loaded on Jurkat A2.1/K^{b} cells. The number of specific spots (delta versus set-up with unloaded Jurkat A2.1/K^{b} cells) is given by the bars

FIG. 6: Th1 responses obtained after heterologous protein prime/MVA boost using non-adjuvanted or aluminium formulated protein compared to immunization with MVA only. Cumulative amount of specific IFN-y spots in CD4+ spleen cells direct ex vivo, after stimulation with HLA-DR-restricted HBV epitopes and PADRE, loaded on syngeneic spleen cells. The number of specific spots (delta) is given by the bars.

FIG. 7: Th2 responses obtained after heterologous protein prime/MVA boost using non-adjuvanted or aluminium formulated protein compared to immunization with MVA only. Cumulative amount of specific IL-5. spots in CD4+ spleen cells direct ex vivo, after stimulation with HLA-DR-restricted HBV epitopes and PADRE, loaded on syngeneic spleen cells. The number of specific spots (delta) is given by the bars.

FIG. 8: CTL responses obtained after heterologous protein prime/MVA boost using various doses of MVA for boost and compared to MVA prime/protein boost. Cumulative amount of specific IFN-γ spots in CD8+ spleen cells direct ex vivo, after stimulation with 6 HLA-A2-restricted HBV epitopes, loaded on Jurkat A2.1/K^{b} cells. The number of specific spots (delta versus set-up with unloaded Jurkat A2.1/K^{b} cells) is given by the bars.

FIG. 9: Th1 responses obtained after heterologous protein prime/MVA boost using various doses of MVA for boost and compared to MVA prime/protein boost. Cumulative amount of specific IFN-γ spots in CD4+ spleen cells direct ex vivo, after stimulation with HLA-DR-restricted HBV epitopes and PADRE, loaded on syngeneic spleen cells. The number of specific spots (delta) is given by the bars.

FIG. 10: Th2 responses obtained after heterologous protein prime/MVA boost using various doses of MVA for boost and compared to MVA prime/protein boost. Cumulative amount of specific IL-5 spots in CD4+ spleen cells direct ex vivo, after stimulation with HLA-DR-restricted HBV epitopes and PADRE, loaded on syngeneic spleen cells. The number of specific spots (delta) is given by the bars.

FIG. 11: CTL responses obtained after heterologous protein prime/MVA boost and heterologous protein prime/DNA boost. Cumulative amount of specific IFN-γ spots in CD8+ spleen cells direct ex vivo, after stimulation with 6 HLA-A2-restricted HBV epitopes, loaded on Jurkat A2.1/K^{b} cells. The number of specific spots (delta versus set-up with unloaded Jurkat A2.1/K^{b} cells) is given by the bars.

FIG. 12: Th1 responses obtained after heterologous protein prime/MVA boost and heterologous protein prime/DNA boost. Cumulative amount of specific IFN-γ spots in CD4+ spleen cells direct ex vivo, after stimulation with HLA-DR-restricted HBV epitopes and PADRE, loaded on syngeneic spleen cells. The number of specific spots (delta) is given by the bars.

FIG. 13: Th2 responses obtained after heterologous protein prime/MVA boost and heterologous protein prime/DNA boost. Cumulative amount of specific IL-5 spots in CD4+ spleen cells direct ex vivo, after stimulation with HLA-DR-restricted HBV epitopes and PADRE, loaded on syngeneic spleen cells. The number of specific spots (delta) is given by the bars.

FIG. 14: CTL responses obtained by repeated cycles of heterologous protein prime/MVA boost. Cumulative amount of specific IFN-γ spots in CD8+ spleen cells direct ex vivo, after stimulation with 6 HLA-A2-restricted HBV epitopes, loaded on Jurkat A2.1/K^{b} cells. The number of specific spots (delta versus set-up with unloaded Jurkat A2.1/K^{b} cells) is given by the bars.

FIG. 15: A. Amino acid sequence of the HBV polyepitope protein.

B. Amino acid sequence of the HCV polyepitope protein.

FIG. 16: Nucleic acid sequence encoding the HBV polyepitope protein with linker and tag his6.

FIG. 17: Nucleic acid sequence encoding the HBV polyepitope protein with linker and tag LHH-11.

FIG. 18: Nucleic acid sequence encoding the HCV polyepitope protein with linker and tag LHH-11.

FIG. 19: Restriction map of plasmid pAcI (ICCG1396).

FIG. 20: Nucleic acid sequence of the plasmid pAcI (1-4947 bps).

FIG. 21: Restriction map of the plasmid pcI857 (ICCG167).

FIG. 22: Nucleic acid sequence of the plasmid pcI857 (1-4182 bps).

FIGS. 23A and B: CTL responses after immunization of HLA-A24/K^{b} or HLA-A11/K^{b} transgenic×Balb/C mice with the HCV polyepitope protein as prime and plasmid DNA as boost. Cumulative amount of specific IFNg spots in CD8⁺ spleen cells direct ex vivo, after stimulation with resp. HLA-A24-restricted HCV epitopes, loaded on LCL721.221HLA-A24/H-2K^{b} cells (FIG. 23A) and HLA A-11-restricted HCV epitopes loaded on LCL721.221HLA-A11/H-2K^{b} cells (FIG. 23B). The number of specific spots (delta) is given by the bars.

FIG. 24: HTL type 1 response after immunization of HLA-A02.1/K^{b} transgenic×Balb/C mice with the HCV polyepitope protein as prime and plasmid DNA as boost. Cumulative amount of specific IFNg spots in CD4⁺ spleen cells direct ex vivo, after stimulation with HLA-DR-restricted HCV epitopes and PADRE, presented by irradiated syngeneic spleen cells. The number of specific spots (delta) is given by the bars.

### DETAILED DESCRIPTION OF THE INVENTION

Unless defined otherwise, all technical and scientific terms used herein have the same meaning as commonly understood by one of ordinary skill in the art. All publications mentioned herein are incorporated by reference.

The present invention relates to methods of vaccination for the effective generation of an antigen-specific immune response in a mammal, preferably a human. Specifically, the present invention relates to prime boost immunization regimens for the generation of a cellular immune response, and more specific a Cytotoxic T Lymphocyte (CTL) and optionally a T Helper Lymphocyte (HTL) response.

The method of the present invention is effective in treating or preventing disease. Many diseases have specific antigens associated with the disease state. Such antigens or epitopes of these antigens are crucial to immune recognition and ultimate elimination or control of the disease in a patient. The present invention provides a vaccine approach based on a prime boost treatment regimen including a protein formulation and this more particular for polyepitope vaccines. It has been demonstrated that the use of a polypeptide comprising a polyepitope construct is especially suited for use as a priming composition in a heterologous prime boost treatment regimen. Surprisingly it was found that, when using the polypeptide as a priming agent, no adjuvant was needed to induce a very high Cytotoxic T Lymphocyte (CTL) response. Moreover, these high CTL responses were obtained without the aid of particle formation promoting proteins and/or carrier proteins.

In a first embodiment, the present invention envisages the use of a polypeptide comprising a polyepitope construct comprising at least two CTL epitopes for the manufacture of a medicament for priming a T cell response against at least one target antigen in a prime boost treatment regimen. More particular, the polypeptide is not linked to or combined with a CTL response inducing adjuvant, a particle formation promoting protein and a carrier protein. In a specific embodiment, the polypeptide is formulated in an aluminium-containing adjuvant. There are three general types of aluminum-containing adjuvants:
Aluminum hydroxide,
Aluminum phosphate, and
Potassium aluminum sulfate (often called “Alum”).

Alum is the adjuvant mostly used for human vaccines. Aluminum-containing adjuvants are known to induce strong antibody (Ab) responses but recognized not to promote or even counter-act the induction of CTL (HogenEsch, 2002; Gupta et al., 1995; Fernando et al., 1998). Immunity against many diseases depends fully or partly on CTL responses and in animal models of tuberculosis, immunisation with alum as an adjuvant even appears to worsen the disease. However, it has been found in the present invention that the use of an aluminium-containing adjuvant has no adverse effect on the CTL inducing activity of the polypeptide as described herein. In fact the high CTL responses are retained if the protein is formulated on alum.

The term “construct” as used herein generally denotes a composition that does not occur in nature. As such, the “polyepitope construct” of the present invention does not encompass a wild-type full-length protein but includes a chimeric protein containing isolated epitopes from at least one protein, not necessarily in the same sequential order as in nature. Said epitopes are “isolated” or “biologically pure”. The term “isolated” refers to material that is substantially free from components that normally accompany it as found in its naturally occurring environment. However, it should be clear that the isolated epitope of the present invention might comprise heterologous cell components or a label and the like. An “isolated” epitope refers to an epitope that does not include the neighbouring amino acids of the whole sequence of the antigen or protein from which the epitope was derived.

With regard to a particular amino acid sequence, an “epitope” is a set of amino acid residues which is involved in recognition by a particular immunoglobulin, or in the context of T cells, those residues necessary for recognition by T cell receptor proteins and/or Major Histocompatibility Complex (MHC) molecules.

The term “peptide” designates a series of amino acids, connected one to the other, typically by peptide bonds between the amino and carboxyl groups of adjacent amino acids.

In a preferred embodiment, the T cell response comprises a Cytotoxic T Lymphocyte (CTL) response. The term “Cytotoxic T Lymphocyte (CTL) response” as used herein refers to a specific cellular immune response mediated by CD8+ cells. This specific cellular immune response can be e.g. the production of specific cytokines such as IFN-gamma (IFN-γ, IFN-g) (measured e.g. by ELISPOT or intracellular FACS), degranulation (measured e.g. by a granzyme-b specific ELISPOT), or cytolytic activity (e.g. measured by a ⁵¹Cr-release assay). Alternatively the antigen specific CD8+ cell can be detected directly by e.g. the use of tetramers.

CTL epitopes have been identified and can be found in literature for many different diseases. It is possible to design epitope strings to generate a CTL response against the “target antigen”, i.e. any chosen antigen that contains at least one of such epitopes. As used herein, the term “antigen” relates to a (complete) protein derived from a pathogen. As understood herein, a “pathogen” is any agent capable of causing disease. It is the aim of the present invention to provide a method of immunising against diseases in which CTL responses play a protective role. Such diseases include but are not limited to infections, i.e. bacterial, protozoan, yeast or viral infection. In a specific embodiment, the infection is an intracellular infection. Such infection is caused by intracellular pathogens including but not limited to mycobacteria, Chlamydia, Legionella, malaria parasites, Aspergillus, Candida, poxviruses, the hepatitis C virus (HCV), the hepatitis B virus (HBV), the human papilloma virus (HPV), the Human Immunodeficiency virus (HIV), influenza, Epstein-Barr virus (EBV), cytomegalovirus (CMV), members of the (human) herpes virus family, measles, dengue and HTLV.

Representative examples of proteins as suitable antigens used in the prevention or treatment of disease are described in the literature and well known to the skilled person. For example, mycobacterial antigens include Mycobacteria tuberculosis proteins from the fibronectin-binding antigen complex (Ag 85). Examples of suitable malaria parasite antigens include the circumsporozoite protein of Plasmodium falciparum. For HIV, particularly preferred antigens include the HIV gag and env proteins (gp-120, p17, gp-160 antigens). The hepatitis B virus presents several different antigens including among others, three HB “Surface” antigens (HBsAgs), an HBcore antigen (HBcAg), an HB e-antigen (HBeAg), and an HB x-antigen (HBxAg).

Also presented by HBV are polymerase (“HBV pol”), ORF 5, and ORF 6 antigens. Preferred immunogenic portion(s) of hepatitis C (HCV) may be found in the UTR, Core, E1, E2, p7 and NS3-NS5 regions. For HPV, immunogenic portions are present in or represented by the L1, L2, E1, E2, E4, E5, E6 and E7 proteins.

According to a specific embodiment of the invention, the pathogen is a virus, the infection is a viral infection and the antigen is a viral protein. More particular, the viral antigen is obtained from HBV, HCV, HPV or HIV and the viral infection is a HBV, HCV, HPV or HIV infection.

The epitopes are of a certain length and bind to a molecule functioning in the immune system, preferably a HLA class I and a T-cell receptor. The epitopes in a polyepitope construct can be HLA class I epitopes and optionally HLA class II epitopes. HLA class I epitopes are referred to as CTL epitopes and HLA class II epitopes are referred to as HTL epitopes. Some polyepitope constructs can have a subset of HLA class I epitopes and another subset of HLA class II epitopes. A CTL epitope usually consists of 13 or less amino acid residues in length, 12 or less amino acids in length, or 11 or less amino acids in length, preferably from 8 to 13 amino acids in length, most preferably from 8 to 11 amino acids in length (i.e. 8, 9, 10, or 11). A HTL epitope consists of 50 or less amino acid residues in length, and usually from 6 to 30 residues, more usually from 12 to 25, and preferably consists of 15 to 20 (i.e. 15, 16, 17, 18, 19, or 20) amino acids in length. The polyepitope construct of the present invention preferably includes 2 or more, 5 or more, 10 or more, 13 or more, 15 or more, 20 or more, or 25 or more CTL epitopes. More specific, the polyepitope construct comprises at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60 or more CTL epitopes.

The polypeptide of the present invention is not linked to or included within a “particle formation promoting” protein, and not linked to or combined with a “carrier protein”. A “particle formation promoting protein” enables the polypeptide to form particles. A “carrier protein” is a protein that transports the polypeptide across intracellular compartments or in extracellular fluids (e.g. in the blood) or else across the cell membrane. Said proteins can be seen as flexible epitope delivery systems and can be administered without adjuvant by various routes leading to strong CTL responses against the included epitopes. Examples of particle formation promoting proteins or carrier proteins are well known to the skilled person and include but are not limited to Ty-VLP, HBsAg, recombinant detoxified adenylate cyclase (CyaA), Shiga toxin, B subunit of E. coli heat-labile toxin (EtxB), Neisseria P64K, pseudomonas endotoxin, anthrax lethal factor, OmpA from Klebsiella pneumoniae, Pseudomonas OprI, OmpA from Haemophilus influenza, and recombinant parvovirus-like particle (PPV-VLP).

Furthermore, the polypeptide of the present invention is not linked to or combined with an adjuvant generally known as promoting induction of CTL responses. Identification of such adjuvant includes performing standard immunogenicity testing for detection of CTL responses induced by the polypeptide in the presence and absence of the adjuvant under the same conditions. In case CTL responses are induced by the polypeptide in the absence of the adjuvant and if significantly higher (i.e. p<0.05) responses are seen with the adjuvanted polypeptide, the adjuvant is considered a CTL response inducing adjuvant.

The following strategy can be utilized to evaluate CTL immunogenicity:

Immunization of subjects such as humans, HLA transgenic mice (see, e.g., Wentworth et al., 1996; Alexander et al., 1997), primates or other mammals having MHC that resembles HLA Class I. In this method, (poly)peptides (in the presence or absence of an adjuvant) are administered to the subjects. Eleven to 14 days following immunization, splenocytes are removed. Cells are cultured in vitro in the presence of test (poly)peptide for approximately one week and peptide-specific CTL's are detected using, e.g., a ⁵¹Cr-release assay involving peptide-sensitized target cells and/or target cells expressing endogenously generated antigen. Alternatively, cells are incubated overnight together with peptide-loaded APC in the IFNg ELISPOT assay for the quantitation of peptide-specific single CD8+ T-cells releasing interferon gamma upon stimulation.

Examples of CTL promoting adjuvants are well known to the skilled person and include, but are not limited to, TLR agonist such as imiquimod, resiquimod, bacterial DNA-based molecules such as ISS (Dynavax) or CpG (Coley Pharmaceuticals), dsRNA molecules, and lipid A or MPL or their synthetic analogues or mimetics such as RC-529 or E6020, may be used either alone or in combinations. Furthermore, immunostimulatory cytokines such as IL-2, GM-CSF or IFN-y, GM-CSF can be used to promote CTL. Other examples include, N-acetyl-muramyl-L-threonyl-D-isoglutamine as described in U.S. Pat. No. 4,606,918, N-acetyl-normuramyl-L-alanyl-D-isoglutamine, N-acetylmuramyl-L-alanyl-D-isoglutamyl-L-alanine2-(1′2′dipalmitoyl-sn-glycero-3-hydroxyphosphoryloxy) ethylamine, RIBI (ImmunoChem Research Inc., Hamilton, Mont., USA) which contains monophosphoryl lipid A, detoxified endotoxin, trehalose-6,6-dimycolate, and cell wall skeleton (MPL+TDM+CWS) in a 2% squalene/Tween 80 emulsion. Any of the three components MPL, TDM or CWS may also be used alone or combined 2 by 2. Additional examples are adjuvants such as Stimulon (Cambridge Bioscience, Worcester, Mass., USA), SAF-1 (Syntex), as well as adjuvants such as combinations between QS21 and 3-de-O-acetylated monophosphoryl lipid A (WO94/00153) or MPL (AS02, GSK), or MF-59 (Chiron), or poly[di(carboxylatophenoxy)phosphazene] based adjuvants (Virus Research Institute), or blockcopolymer based adjuvants such as Optivax (Vaxcel, Cytrx) or Incomplete Freund's Adjuvant (IFA), Complete Freund's Adjuvant (CFA), or Gerbu preparations (Gerbu Biotechnik). Further examples are Immune Stimulating Complexes together with saponins (ISCOMS or ISCOMATRIX™). CTL promoting adjuvants also cover preparations emulsified or encapsulated in liposomes for enhancing adjuvant effect, or bacterial preparations such as BCG (Bacillus Calmette-Guerin) or Corynebacterium parvum.

In a further embodiment, the T cell response comprises a T Helper response. Accordingly, the polyepitope construct of the invention further comprises one or more HTL (T Helper) epitopes. The at least one HTL epitope can be derived from any target antigen. As such, the polyepitope construct of the present invention optionally comprises at least 1, 2, 3, 4, 5, 6, 7, 8, 9, 10, 11, 12, 13, 14, 15, 16, 17, 18, 19, 20, 25, 30, or more HTL epitopes. In a preferred embodiment, the polyepitope construct of the present invention comprises the universal T cell epitope called PADRE® (Epimmune, San Diego; described, for example in U.S. Pat. No. 5,736,142 or International Application WO95/07707, which are enclosed herein by reference). A ‘PanDR binding peptide or PADRE® peptide” is a member of a family of molecules that binds more that one HLA class II DR molecule. The pattern that defines the PADRE® family of molecules can be thought of as an HLA class II supermotif. PADRE® binds to most HLA-DR molecules and stimulates in vitro and in vivo human helper T lymphocyte (HTL) responses.

In another embodiment, the polyepitope construct of the present invention comprises T-help epitopes derived from the same pathogen as the CTL epitopes.

Alternatively T-help epitopes can be used from universally used vaccines such as tetanos toxoid. It may also be useful to include B cell epitopes in the polyepitope construct for stimulating B cell responses and antibody production.

In a further embodiment, the use of the polypeptide of the priming composition as described herein is directed against a virus. More specific, the polyepitope construct contains 2, 3, 4, 5, 10, 15, 20 or more epitopes derived from a virus. More particular, two or more CTL epitopes in the polyepitope construct are derived from the Hepatitis B virus (HBV), and more specifically from the HBV Core protein, the HBV Polymerase protein and/or the HBV Envelope protein. Even more particular, two or more CTL epitopes are selected from the list of epitopes given in Table 1. In a preferred embodiment, the polyepitope construct as described herein comprises all the CTL epitopes given in Table 1. Optionally, the polyepitope construct furthermore comprises one or more HTL epitopes. More particular, at least one HTL epitope is selected from the list of epitopes given in Table 2. In a preferred embodiment, the polyepitope construct as described herein comprises all the HTL epitopes given in Table 2.

In an alternative embodiment, the two or more CTL epitopes in the polyepitope construct are derived from the Hepatis C virus (HCV), and more specifically from the HCV CORE, E1, E2, NS3, NS4 and/or NS5 protein. Even more specific, the two or more CTL epitopes in the polyepitope construct include one or more HCV epitopes selected from the list of epitopes given in Table 3. In a particular embodiment, the polyepitope construct as described herein comprises all the CTL epitopes given in Table 3. Optionally, the polyepitope construct of the present invention furthermore comprises one or more HTL epitopes selected from the list of epitopes given in Table 4. In a preferred embodiment, the polyepitope construct as described herein comprises all the HTL epitopes given in Table 4.

The epitopes of the polyepitope construct are directly or indirectly linked to one another. More specific, two or more of the epitopes (either CTL and/or HTL) are either contiguous or are separated by a linker or one or more spacer amino acids. “Link” or “join” refers to any method known in the art for functionally connecting peptides (direct or via a linker), including, without limitation, recombinant fusion, covalent bonding, non-covalent bonding, disulfide bonding, ionic bonding, hydrogen bonding, polymerization, cyclization, electrostatic bonding and connecting through a central linker or carrier. Polymerization can be accomplished for example by reaction between glutaraldehyde and the —NH2 groups of the lysine residues using routine methodology. More particular, the polyepitope construct of the present invention is a recombinant string of two or more epitopes.

In a specific embodiment, the polyepitope construct of the present invention further comprises one or a plurality of spacer amino acids between two or more epitopes. More specific, the polyepitope construct comprises 1 to 9, and more preferably 1 to 5 spacer amino acids, i.e. 1, 2, 3, 4 or 5 spacer amino acids between two or more, or all, of the epitopes in the construct. A “spacer” refers to a sequence that is inserted between two epitopes in a polyepitope construct to prevent the occurrence of junctional epitopes (an epitope recognized by the immune system, not present in the target antigen, and only created by the man-made juxtaposition of epitopes), or to facilitate cleavage between epitopes and thereby enhance epitope presentation.

To develop polyepitope constructs using the epitopes of the present invention, said epitopes can be sorted and optimized using a computer program or, for fewer epitopes, not using a computer program. “Sorting epitopes” refers to determining or designing an order of the epitopes in a polyepitope construct.

“Optimizing” refers to increasing the antigenicity of a polyepitope construct having at least one epitope pair by sorting epitopes to minimize the occurrence of junctional epitopes, and inserting a spacer residue (as described herein) to further prevent the occurrence of junctional epitopes or to provide a flanking residue. As described herein, a “flanking residue” is a residue that is positioned next to an epitope. A flanking residue can be introduced or inserted at a position adjacent to the N-terminus (N+1) or the C-terminus (C+1) of an epitope. An increase in immunogenicity or antigenicity of an optimized polyepitope construct is measured relative to a polyepitope construct that has not been constructed based on the optimization parameters by using assays known to those skilled in the art, e.g. assessment of immunogenicity in HLA transgenic mice, ELISPOT, tetramer staining, ⁵¹Cr release assays, and presentation on antigen presenting cells in the context of MHC molecules. The process of optimizing polyepitope constructs is given e.g. in WO01/47541 and WO04/031210 (Pharmexa Inc. et al.; incorporated herein by reference). It is preferred that spacers are selected by concomitantly optimizing epitope processing and preventing junctional motifs. The “spacer amino acid” or “spacer peptide” is typically comprised of one or more relatively small, neutral molecules, such as amino acids or amino acid mimetics, which are substantially uncharged under physiological conditions. For example, spacers flanking HLA class II epitopes preferably include G (Gly), P (Pro), and/or N (Asn) residues. A particularly preferred spacer for flanking a HLA class II epitope includes alternating G and P residues, for example, (GP)n, (PG)n, (GP)nG, (PG)nP, and so forth, where n is an integer between 1 and 10, preferably 2 or 3, and where a specific example of such a spacer is GPGPG (SEQ ID NO 103). For separating class I epitopes, or separating a class I and a class II epitope, the spacers are typically selected from, e.g., A (Ala), N (Asn), K (Lys), G (Gly), L (Leu), I (Ile), R (Arg), Q (Gln), S (Ser), C (Cys), P (Pro), T (Thr), or other neutral spacers of nonpolar amino acids or neutral polar amino acids, though polar residues could also be present. A preferred spacer, particularly for HLA class I epitopes, comprises 1, 2, 3 or more consecutive alanine (A), lysine (K) or asparagine (N) residues, or a combination of K (Lys) and A (Ala) residues, e.g. KA, KAA or KAAA, or a combination of N (Asn) and A (Ala) residues, e.g. NA, NAA or NAAA, or a combination of G (Gly) and A (Ala) residues, e.g. GA or GAA. The present invention is thus directed to a polypeptide comprising a polyepitope construct as described herein, and wherein the epitopes in the construct are separated by one or more spacer amino acids. In a preferred embodiment, the one or more spacer amino acids are selected from the group consisting of: K, R, N, Q, G, A, S, C, G, P and T.

The peptides can be in their natural (uncharged) forms or in forms which are salts, and either free of modifications such as glycosylation, side chain oxidation, or phosphorylation or containing these modifications. Also included in the definition are peptides modified by additional substituents attached to the amino acids side chains, such as glycosyl units, lipids, or inorganic ions such as phosphates, as well as modifications relating to chemical conversions of the chains, such as oxidation of sulfhydryl groups. Thus, “peptide” or its equivalent terms is intended to include the appropriate amino acid sequence referenced, and may be subject to those of the foregoing modifications as long as its functionality is not destroyed.

In a specific embodiment, the cysteine residues of the peptides in the polyepitope construct are reversibly blocked. Preferably, the cysteine residues are sulphonated.

Moreover, the present invention also contemplates a polyepitope construct comprising or consisting of multiple repeats or combinations of any of the epitopes of the present invention. The polyepitope construct can exist as a homopolymer comprising multiple copies of the same (combination of) peptide(s), or as a heteropolymer of various peptides. Polymers have the advantage of increased immunological reaction and, where different peptide epitopes are used to make up the polymer, the additional ability to induce HTL's and/or CTLs that react with different antigenic determinants of the pathogenic organism targeted for an immune response.

In a further embodiment, the polyepitope protein is linked to a metal affinity tag. Such tags are well known to the skilled person and include but are not limited to a hexahistidine tag (his6) or a tag consisting of the sequence

| | |
|---|---|
| HHMFHHHWWHHHMWHHH. | (SEQ ID NO 97; LHH11) |

A polypeptide comprising multiple epitopes or a polyepitope construct can be generated synthetically, recombinant (Thomson et al., 1996), or via cleavage from the native source (Alexander et al., 2002). Said polypeptide can be expressed as one protein. In order to carry out the expression of the polypeptide in bacteria, in eukaryotic cells (including yeast) or in cultured vertebrate hosts such as Chinese Hamster Ovary (CHO), Vero cells, RK13, COS1, BHK, and MDCK cells, or invertebrate hosts such as insect cells, the following steps are carried out:
transformation of an appropriate cellular host with a recombinant vector, or by means of adenoviruses, influenza viruses, BCG, and any other live carrier systems, in which a nucleotide sequence coding for one of the polypeptides of the invention has been inserted under the control of the appropriate regulatory elements, particularly a promoter recognized by the polymerases of the cellular host or of the live carrier system and in the case of a prokaryotic host, an appropriate ribosome binding site (RBS), enabling the expression in said cellular host of said nucleotide sequence,
culture of said transformed cellular host under conditions enabling the expression of said insert.

In a preferred embodiment, the polypeptide comprising the polyepitope construct of the present invention is the result of bacterial or yeast expression. More preferably, the bacterial expression is performed in E. coli species.

In another aspect of the invention, the isolated polypeptide according to the invention is the product of expression in a yeast cell. More particularly, the isolated polypeptide according to the invention is the product of expression in a cell of strains of Saccharomyces, such as Saccharomyces cerevisiae, Saccharomyces kluyveri, or Saccharomyces uvarum, Schizosaccharomyces, such as Schizosaccharomyces pombe, Kluyveromyces, such as Kluyveromyces lactis, Yarrowia, such as Yarrowia lipolytica, Hansenula, such as Hansenula polymorpha, Pichia, such as Pichia pastoris, Aspergillus species, Neurospora, such as Neurospora crassa, or Schwanniomyces, such as Schwanniomyces occidentalis, or mutant cells derived from any thereof. More specifically, the polypeptide according to the invention is the product of expression in a Hansenula cell or Saccharomyces cerevisiae cell.

The polypeptide or protein can be purified by methods well known to the person skilled in the art.

According to the present invention, the priming composition or priming agent used in the prime boost treatment regimen comprises the polypeptide containing a polyepitope construct as described herein.

The boosting composition or boosting agent may be provided in a variety of different forms. Specifically, the boosting composition is a vector. More specific, the vector is plasmid DNA or a viral vector.

In a particular embodiment, the present invention relates to the use of a polypeptide comprising a polyepitope construct comprising at least two CTL epitopes for the manufacture of a medicament for priming a T cell response against at least one target antigen in a prime boost treatment regimen, comprising the steps of:
a. administering the polypeptide as a priming composition; and
b. administering a boosting composition comprising a vector encoding one or more CTL epitopes of the target antigen, including at least one CTL epitope which is the same as a CTL epitope of the priming composition.

More particularly, the polypeptide is not linked to, not included within and/or not combined with a particle formation promoting protein and/or a carrier protein. Even more particularly, the polypeptide is not combined with a CTL response inducing adjuvant.

Preferably, the vector of the boosting composition comprises one or more CTL epitopes of the target antigen, including at least one isolated CTL epitope which is the same as a CTL epitope of the priming composition. Said at least one CTL epitope can be comprised in a larger protein. More preferably, the epitopes encoded by the vector of the boosting composition are the same as the epitopes of the polyepitope construct of the priming polypeptide. Accordingly, the present invention thus also relates to a vector comprising a polynucleotide encoding a polyepitope construct. In this context it is noted that practically all considerations pertaining to the polypeptide construct described herein apply to the polynucleotide construct. The term “polyepitope construct” when referring to nucleic acids and polynucleotides can be used interchangeably with the terms “minigene” and “polyepitope nucleic acid” and other equivalent phrases, and comprises multiple nucleic acid epitopes that encode peptides of certain length that can bind to a molecule functioning in the immune system, preferably a HLA class I or a HLA class II and a T-cell receptor. All disclosures herein with regard to epitopes comprised in an amino acid construct apply mutatis mutandis to the nucleic acid epitopes comprised in a polynucleotide or DNA construct.

With regard to a particular nucleic acid sequence, a “nucleic acid epitope” is a set of nucleic acids that encode for a particular amino acid sequence that forms an epitope. DNA and vector related specifications are well known to the person skilled in the art.

In a particular embodiment, the vector is a plasmid, a bacterial, a viral vector or a yeast vector. Preferred bacterial vectors are Salmonella typhi, BCG (Bacille Calmette Guerin; described in Stover et al., 1991) and Listeria. Preferred viral vectors are poxvirus, Alphaviruses (Semliki Forest Virus, Sindbis Virus, Venezuelan Equine Encephalitis Virus (VEE), Herpes simplex Virus (HSV), Kunjin virus, Vesicular Stomatitis Virus (VSV) replication-deficient strains of Adenovirus (human or simian), polyoma vectors (such as SV40 vectors, bovine polyoma), CMV vectors, papilloma virus vectors, influenza virus, measles virus, and vectors derived from Epstein Barr virus. A wide variety of other vectors useful for therapeutic administration or immunization, e.g. lentiviral vectors, retroviral vectors, and the like, will be apparent to those skilled in the art. In a specific embodiment, the vector of the boosting composition is a recombinant vaccinia virus. Preferred yeast vectors are a Hansenula cell or Saccharomyces cerevisiae cell.

Preferably, the vector of the boosting composition is a non-replicating or replication impaired viral vector. The term “non-replicating” or “replication-impaired” as used herein means not capable of replication to any significant extent in the majority of normal mammalian cells or normal human cells. Viruses which are non-replicating or replication-impaired may have become so naturally (i.e. they may be isolated as such from nature) or artificially e.g. by breeding in vitro or by genetic manipulation, for example deletion of a gene which is critical for replication. There will generally be one or a few cell types of non-human origin in which the viruses can be grown, such as CEF cells for MVA. Replication of a virus is generally measured in two ways: 1) DNA synthesis and 2) viral titre.

More precisely, the term “non-replicating or replication-impaired” as used herein and as it applies to poxviruses means viruses which satisfy either or both of the following criteria: 1) exhibit a 1 log (10 fold) reduction in DNA synthesis compared to the Copenhagen strain of vaccinia virus in MRC-5 cells (a human cell line); 2) exhibit a 2 log reduction in viral titre in HELA cells (a human cell line) compared to the Copenhagen strain of vaccinia virus. Examples of poxviruses which fall within this definition are MVA, NYVAC and avipox viruses. It will be evident that vaccinia virus strains derived from MVA, or independently developed strains having the features of MVA which make MVA particularly suitable for use in a vaccine, will also be suitable for use in the invention.

As an example of this approach, MVA is used as a vector to express nucleotide sequences that encode the epitopes of the invention. Upon introduction into a host, the recombinant vaccinia virus expresses the immunogenic peptide, and thereby elicits a host CTL and/or HTL response. Vaccinia vectors, for example Modified Vaccinia Ankara (MVA), and methods useful in immunization protocols are described in, e.g., U.S. Pat. No. 4,722,848 or WO9813500.

In a particular embodiment, the vector of the present invention further comprises one or more regulatory sequences. By “regulatory sequence” is meant a polynucleotide sequence that contributes to or is necessary for the expression of an operably associated nucleic acid or nucleic acid construct in a particular host organism. The regulatory sequences that are suitable for eukaryotes, for example, include a promoter (e.g. CMV promoter), optionally an enhancer sequence, introns with functional splice donor and acceptor sites, a Kozak consensus sequence, signal sequences (e.g. Ig kappa light chain signal sequence), an internal ribosome entry site (IRES), and polyadenylation signals (e.g. SV40 early poly-A signal). Other specific examples of regulatory sequences are described herein and otherwise known in the art. A typical expression cassette thus contains all necessary regulatory elements required for efficient transcription and translation of the gene.

Suitable promoters are well known in the art and described, e.g., in Sambrook et al., Molecular cloning, A Laboratory Manual (2^{nd} ed. 1989) and in Ausubel et al, Current Protocols in Molecular Biology (1994). Eukaryotic expression systems for mammalian cells are well known in the art and are commercially available. Such promoter elements include, for example, cytomegalovirus (CMV), Rous sarcoma virus long terminal repeats (RSV LTR) and Simian Virus 40 (SV40). See, e.g., U.S. Pat. Nos. 5,580,859 and 5,589,466 for other suitable promoter sequences.

In addition to a promoter sequence, the expression cassette can also contain a transcription termination region downstream of the structural gene to provide for efficient termination. The termination region may be obtained from the same gene as the promoter sequence or may be obtained from different genes.

Additional vector modifications may be desired to optimize epitope expression and immunogenicity. In some cases, introns are required for efficient gene expression, and one or more synthetic or naturally-occurring introns could be incorporated into the transcribed region. The inclusion of mRNA stabilization sequences and sequences for replication in mammalian cells may also be considered for increasing epitope expression. In addition, immunostimulatory sequences (ISSs or CpGs) appear to play a role in the immunogenicity of nucleic acid vaccines. These sequences may be included in the vector, outside the polynucleotide coding sequence, if desired to enhance immunogenicity. In some embodiments, a bi-cistronic expression vector which allows production of both the CTL epitopes and a second protein (included to enhance or decrease immunogenicity) can be used. Examples of proteins or polypeptides that could beneficially enhance the immune response if co-expressed include cytokines (e.g., IL-2, IL-12, GM-CSF), cytokine-inducing molecules (e.g., LeIF), costimulatory molecules, or for HTL responses, pan-DR binding proteins (PADRE®, Epimmune, San Diego, Calif.).

Helper (HTL) epitopes can be joined to intracellular targeting signals and expressed separately from expressed CTL epitopes; this allows direction of the HTL epitopes to a cell compartment different than that of the CTL epitopes. If required, this could facilitate more efficient entry of HTL epitopes into the HLA class II pathway, thereby improving HTL induction. In contrast to HTL or CTL induction, specifically decreasing the immune response by co-expression of immunosuppressive molecules (e.g. TGF-P) may be beneficial in certain diseases.

The polypeptide and the vector of the present invention are used in a prime boost regimen. More particular, the regimen is a heterologous prime boost regimen. The term “heterologous” as used herein refers to a different presentation format, i.e. protein versus vector, of the epitopes in the priming versus the boosting agent. It is to be understood that the term “prime boost regimen” or “prime boost treatment regimen” refers to the administration of the compounds in a certain order and with a certain time interval. A “prime boost regimen” or “prime boost treatment regimen” can consist of one or multiple, i.e. two, three four or more, prime boost cycles. The term “prime” or “priming” as used herein refers to the composition administered first in a prime boost cycle. The term “boost” or “boosting” as used herein refers to the composition administered, in a prime boost cycle, with a certain time interval after the prime or priming. It is to be understood that the prime may consist of more than one administration (separated in time and/or site of injection) of the same composition. Similarly, it is to be understood that the boost may consist of more than one administration (separated in time and/or site of injection) of the same composition. In its broadest interpretation, the time interval between prime and boost in one cycle or between two cycles can go from one day to 24 weeks or even up to 1 year. More specific, the time interval between the administrations of prime and boost within 1 cycle includes 1 week, 2 weeks, 3 weeks, 4 weeks, 5 weeks, 6 weeks, 7 weeks, 8 weeks, 9 weeks, 10 weeks, 11 weeks or 12 weeks and any time interval in between. Similarly, the time interval between two prime boost cycles includes 1 week, 2 weeks, 3 weeks, 6 weeks, 8 weeks, 10 weeks, 15 weeks, 20 weeks, 30 weeks, 40 weeks, and up to one year. Specifically, the time interval between the administrations of the priming composition and the boosting composition is from 1 to 12 weeks. More specific, the time interval is 1 to 4 weeks. Even more specific, the time interval is 2 or 3 weeks, plus or minus 1 to 3 days. Due to specific circumstances (e.g. illness), it may not be possible to stick at exact 2 or 3 weeks interval, therefore a deviation of 1 to 3 days is permissible.

It has been demonstrated by the present invention that the application of a certain treatment regimen will improve the induction of the desired immune response. If the prime-boost cycle is repeated multiple times (2 or more), a higher CTL response is obtained. In case of repeated prime-boost cycles the number of administrations for each of the prime and boost may be varied between the cycles.

In a particular embodiment, the present invention relates to the use of a polypeptide comprising a polyepitope construct comprising at least two CTL epitopes for the manufacture of a medicament for priming a Cytotoxic T Lymphocyte (CTL) immune response against at least one target antigen in a prime boost treatment regimen, wherein the polypeptide is not linked to, included within or combined with a compound selected from the group consisting of: a particle formation promoting protein, a carrier protein and a CTL response inducing adjuvant, and wherein the prime boost treatment regimen comprises the following steps (in the given order):
a. a prime with 1, 2 or 3 polypeptide administrations;
b. a boost with 1 or 2 vector administrations.

In said treatment regimen, the polypeptide and/or vector are administered in an “effective amount”. An “effective amount” of the polypeptide or vector is referred to as an amount required and sufficient to elicit an immune response, specifically a T cell response, and more especially a CTL response, preferably determined subsequent to the prime boost cycle. It will be clear to the skilled artisan that the immune response sufficiently broad and vigorous to provoke the effects envisaged by the prime boost cycle may require successive (in time) immunizations with the polypeptide and/or vector as part of a vaccination scheme or vaccination schedule. The “effective amount” may vary depending on the health and physical condition of the individual to be treated, the age of the individual to be treated (e.g. dosing for infants may be lower than for adults) the taxonomic group of the individual to be treated (e.g. human, non-human primate, primate, etc.), the capacity of the individual's immune system to mount an effective immune response, the degree of protection desired, the formulation of the composition, the treating doctor's assessment, the strain of the infecting pathogen and other relevant factors. It is expected that the effective amount of the composition will fall in a relatively broad range that can be determined through routine trials. The dosage may be administered in a single administration schedule or in a multiple administration schedule. In a multiple administration schedule, the total effective amount (or dose) is subdivided and administered at different sites, this within 24 hours, preferably within 8 hours and more preferably within 2 hours. The dosages, routes of administration, and dose schedules are adjusted in accordance with methodologies known in the art.

The polypeptide and vector compositions of this invention can be provided in kit form together with instructions for administration. Typically the kit would include the polypeptide composition as described herein in a container, preferably in unit dosage form and instructions for administration. In a particular embodiment, the present invention provides a kit for inducing a CTL response against at least one target antigen, which kit comprises a priming composition comprising a polypeptide comprising a polyepitope construct comprising two or more CTL epitopes of the target antigen. More particular, the polypeptide is not linked to, combined with or included within a compound selected from the group consisting of: a particle formation promoting protein, a carrier protein and a CTL response inducing adjuvant.

In a further embodiment, the kit also comprises a boosting composition comprising a vector encoding for one or more CTL epitopes of the target antigen, including at least one CTL epitope which is the same as a CTL epitope of the priming composition. The kit includes the vector in a container, preferably in unit dosage form together with instructions for administration. Other kit components that may also be desirable include, for example, a sterile syringe and other desired excipients.

All embodiment relating to the polypeptide and/or the vector of the invention are applicable to the kit comprising said compounds.

In another aspect the invention provides a method for generating an immune response against at least one target antigen, which method comprises administering at least one dose of the priming composition, followed by at least one dose of the boosting composition according to the invention. More particular, the immune response is a T cell response. Even more particular, the immune response is a CTL and optionally a HTL response.

All embodiments described herein relating to the polypeptide and the vector of the invention as well as the administration pattern are applicable to the method using said compounds.

The polypeptide of the present invention is preferably included in a composition, more specifically a pharmaceutical composition. In a particular embodiment, the present invention relates to the use of a pharmaceutical composition for priming a CTL response against at least one target antigen, said composition comprising a polypeptide comprising a polyepitope construct as described herein, and optionally a pharmaceutical acceptable excipient. More particularly, said pharmaceutical composition is a vaccine composition. Even more particular, said pharmaceutical composition is a therapeutic vaccine composition. Alternatively, said pharmaceutical composition can also be a prophylactic vaccine composition. The therapeutic vaccine composition refers to a vaccine composition aimed for treatment of infection and to be administered to patients being infected. The prophylactic vaccine composition refers to a vaccine composition aimed for preventing infection and to be administered to healthy persons who are not yet infected. The pharmaceutical composition may additionally comprise one or more further active substances and/or at least one of a pharmaceutically acceptable excipient such as water, saline, physiological salt solutions, glycerol, ethanol, etc. However, the pharmaceutical composition does not comprise a CTL response inducing adjuvant.

The vector of the present invention is preferably included in a composition, more specifically a pharmaceutical composition. Various art-recognized delivery systems may be used to deliver the vector of the invention into appropriate cells. The vector can be delivered in a pharmaceutically acceptable carrier or as colloidal suspensions, or as powders, with or without diluents. They can be “naked” or associated with delivery vehicles and delivered using delivery systems known in the art.

A “pharmaceutically acceptable vehicle” or “pharmaceutically acceptable carrier” includes vehicles such as water, saline, physiological salt solutions, glycerol, ethanol, etc. Auxiliary substances such as wetting or emulsifying agents, pH buffering substances, preservatives may be included in such vehicles.

Typically, a composition or vaccine is prepared as an injectable, either as a liquid solution or suspension. Injection may be subcutaneous, intramuscular, intravenous, intraperitoneal, intrathecal, intradermal, intraepidermal, or by “gene gun”. Other types of administration comprise electroporation, implantation, suppositories, oral ingestion, enteric application, inhalation, aerosolization or nasal spray or drops. Solid forms, suitable for dissolving in, or suspension in, liquid vehicles prior to injection may also be prepared. A liquid formulation may include oils, polymers, vitamins, carbohydrates, amino acids, salts, buffers, albumin, surfactants, or bulking agents. Any physiological buffer may be used.

After the liquid pharmaceutical composition is prepared, it is preferably lyophilized to prevent degradation and to preserve sterility. Methods for lyophilizing liquid compositions are known to those of ordinary skill in the art. Just prior to use, the composition may be reconstituted with a sterile diluent (Ringer's solution, distilled water, or sterile saline, for example) which may include additional ingredients. Upon reconstitution, the composition is preferably administered to subjects using those methods that are known to those skilled in the art.

The approach known as “naked DNA” is currently being used for intramuscular (IM) administration in clinical trials. To maximize the immunotherapeutic effects of DNA vaccines, an alternative method for formulating purified plasmid DNA may be desirable. A variety of methods have been described, and new techniques may become available. Cationic lipids can also be used in the formulation (see, e.g., as described by WO 93/24640; Mannino & Gould-Fogerite 1988; U.S. Pat. No. 5,279,833; WO 91/06309; and Feigner et al., 1987). In addition, glycolipids, fusogenic liposomes, peptides and compounds referred to collectively as protective, interactive, non-condensing compounds could also be complexed to purified plasmid DNA to influence variables such as stability, intramuscular dispersion, or trafficking to specific organs or cell types. Further examples of DNA-based delivery technologies include facilitated (bupivicaine, polymers, peptide-mediated) delivery, cationic lipid complexes, particle-mediated (“gene gun”) or pressure-mediated delivery (see, e.g., U.S. Pat. No. 5,922,687), DNA formulated with charged or uncharged lipids, DNA formulated in liposomes, emulsified DNA, DNA included in a viral vector, DNA formulated with a transfection-facilitating protein or polypeptide, DNA formulated with a targeting protein or polypeptide, DNA formulated with calcium precipitating agents, DNA coupled to an inert carrier molecule, and DNA formulated with an adjuvant. In this context it is noted that practically all considerations pertaining to the use of adjuvants in traditional vaccine formulation apply to the formulation of DNA vaccines. Detailed disclosures relating to the formulation and use of nucleic acid vaccines are available, e.g. by Donnelly J. J. et al, 1997 and 1997a.

The present invention also relates to a recombinant virus, a bacterial vector, a yeast vector or a plasmid, and a host cell comprising the polynucleotide as described herein. Additional vector modifications may be desired to optimize polynucleotide expression and immunogenicity and are well known to the person skilled in the art.

Other arrangements of the methods and tools embodying the invention will be obvious for those skilled in the art.

It is to be understood that although preferred embodiments, specific constructions and configurations, as well as materials, have been discussed herein for the methods and tools according to the present invention, various changes or modifications in form and detail may be made without departing from the scope and spirit of this invention.

The present invention is illustrated by the following Examples, which should not be understood to limit the scope of the invention to the specific embodiments therein.

TABLE 1
HBV CTL epitopes

| HBV | | |
|---|---|---|
| protein | Amino acid sequence | SEQ ID NO |
| Pol | FLLSLGIHL | 1 |
| Pol | KYTSFPWLL | 2 |
| Env | RFSWLSLLVPF | 3 |
| Pol | FPHCLAFSYM | 4 |
| Pol | LVVDFSQFSR | 5 |
| Env | ILLLCLIFLL | 6 |
| Pol | HTLWKAGILYK | 7 |
| Env | WMMWYWGPSLY | 8 |
| Pol | YPALMPLYACI | 9 |
| Env | WLSLLVPFV | 10 |
| Env | FLLTRILTI | 11 |
| Env | IPIPSSWAF | 12 |
| Core | EYLVSFGVW | 13 |
| Core | LPSDFFPSV | 14 |
| Core | FLPSDFFPSV | 15 |
| Core | DLLDTASALY | 16 |
| Pol | SWPKFAVPNL | 17 |
| Pol | SAICSVVRR | 18 |
| Pol | LSLDVS | 19 |
| Pol | NVSIPWTHK | 20 |
| Pol | GLSRYVARL | 21 |
| Core | STLPETTVVRR | 22 |
| Pol | HPAAMPHLL | 23 |
| Env | RWMCLRRFII | 24 |
| Pol | ASFCGSPY | 25 |
| Pol | YMDDVVLGV | 26 |
| Core | LWFHISCLTF | 27 |
| Pol | TPARVTGGVF | 28 |
| Core | LTFGRETVLEY | 29 |
| Pol | QAFTFSPTYK | 30 |

Pol = polymerase
Env = envelope

TABLE 2
HBV HTL epitopes

| HBV | | |
|---|---|---|
| protein | Amino acid sequence | SEQ ID NO |
| pol | GTSFVYVPSALNPAD | 31 |
| pol | LCQVFADATPTGWGL | 32 |
| pol | RHYLHTLWKAGILYK | 33 |
| core | PHHTALRQAILCWGELMTLA | 34 |
| pol | ESRLVVDFSQFSRGN | 35 |
| pol | PFLLAQFTSAICSVV | 36 |
| env | LVPFVQWFVGLSPTV | 37 |
| pol | LHLYSHPIILGFRKI | 38 |
| pol | SSNLSWLSLDVSAAF | 39 |
| pol | LQSLTNLLSSNLSWL | 40 |
| env | AGFFLLTRILTIPQS | 41 |
| core | VSFGVWIRTPPAYRPPNAPI | 42 |
| pol | VGPLTVNEKRRLKLI | 43 |
| pol | KQCFRKLPVNRPIDW | 44 |
| pol | AANWILRGTSFVYVP | 45 |
| pol | KQAFTFSPTYKAFLC | 46 |
| PADRE | AKFVAAWTLKAAA | 47 |

Pol = polymerase
Env = envelope

TABLE 3
HCV CTL epitopes

| HCV Protein | Amino acid sequence | SEQ ID NO |
|---|---|---|
| NS3 | VATDALMTGY | 48 |
| NS3 | FTDNSSPPAV | 49 |
| NS4B | LVDILAGYGA | 50 |
| NS4A | VLVGGVLAAL | 51 |
| C | YLLPRRGPRL | 52 |
| NS4B | HMWNFISGI | 53 |
| NS5B | TLWARMILM | 54 |
| NS3 | GLLGCIITSL | 55 |
| NS5B | KLQDCTMLV | 56 |
| NS5B | NIIMYAPTL | 57 |
| NS3 | AVCTRGVAK | 58 |
| C | KTSERSQPR | 59 |
| C | STNPKPQRK | 60 |
| NS3 | AAYAAQGYK | 61 |
| NS4B | QYLAGLSTL | 62 |
| NS3 | MYTNVDQDL | 63 |
| NS4B | FWAKHMWNFI | 64 |
| NS3 | VIKGGRHLI | 65 |
| NS3 | CLIRLKPTL | 66 |
| C | LPRRGPRLGV | 67 |
| NS5B | APTLWARMIL | 68 |
| NS5B | LPINALSNSL | 69 |
| C | SFSIFLLAL | 70 |
| NS3 | AYYRGLDVSV | 71 |
| NS5B | YRRCRASGVL | 72 |
| C | DPRRRSRNL | 73 |
| C | GPRLGVRAT | 74 |
| C | LPGCSFSIF | 75 |
| NS3 | LIRLKPTLH | 76 |
| NS3 | HPNIEEVAL | 77 |
| NS3 | FPYLVAYQA | 78 |
| NS3 | AETAGARLV | 79 |
| NS4b | AEQFKQKAL | 80 |
| E2 | DYPYRLWHY | 81 |

TABLE 5
HCV HTL epitopes

| HCV Protein | Amino acid sequence | SEQ ID NO |
|---|---|---|
| NS3 | AVGIFRAAVCTRGVA | 82 |
| NS3 | GYKVLVLNPSVAAT | 83 |
| NS3 | GRHLIFCHSKKKCD | 84 |
| NS3 | CVTQTVDFSLDPTFTIETT | 85 |
| NS3 | TPAETTVRLRAYMNTPGLPV | 86 |
| NS4A | TSTWVLVGGVLAALAA | 87 |
| NS4A | GKPAIIPDREVLYRE | 88 |
| NS4B | GIQYLAGLSTLPGNPA | 89 |
| NS4B | STLLFNILGGWVAAQ | 90 |
| NS4B | EDLVNLLPAILSPGALVV | 91 |
| NS4B | GEGAVQWMNRLIAFAS | 92 |
| NS4B | MNRLIAFASRGNHVSP | 93 |
| NS5B | SMSYTWTGALITPCA | 94 |
| PADRE | AKFVAAWTLKAAA | 47 |

### EXAMPLES

### Example 1

### Generation of a Polyepitope DNA Vector

1.1. HBV

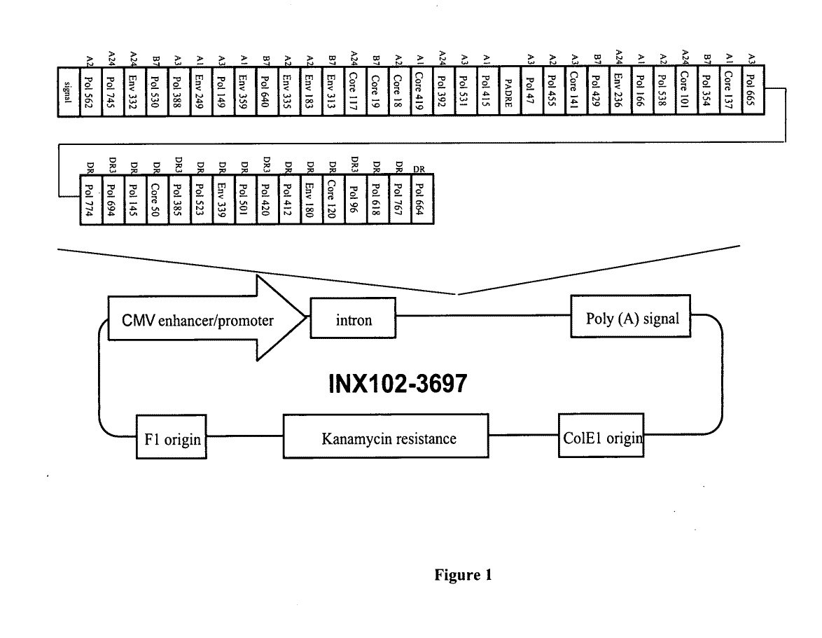

The gene construct GCR-3697 as described in WO04/031210 (Pharmexa Inc.) was assembled using overlapping oligonucleotides in a PCR-based synthesis followed by subcloning into the pMB75.6 DNA plasmid vector (Wilson et al., 2003). Expression of the gene is driven by the CMV-IE promoter (FIG. 1). The DNA sequence was optimized to remove rare human codons and to reduce the ability to form potentially deleterious secondary RNA structures. Plasmid DNA (pDNA) was produced by growth in E. coli Stb12 strain (Invitrogen) in Terrific Broth with kanamycin (25 μg/ml) and purified using Qiagen Endotoxin-Free MegaPrep columns according to the manufacturer's directions (Qiagen USA, Valencia, Calif.). The purified pDNA HBV construct, (INX102-3697), was dissolved in water and stored at −70° C. For immunizations, the pDNA was formulated in 3.4% poly(N-vinyl pyrrolidone), 3 mg/ml ethanol and PBS, pH 7.4 at a concentration of 2 mg/ml.

1.2. HCV

The HCV gene construct was assembled using overlapping oligonucleotides in a PCR-based synthesis followed by subcloning into pMB75.6 DNA plasmid vector (Wilson et al., 2003) using PstI and BamHI restriction sites. A Kozak sequence, a mouse Igk signal sequence (MGMQVQIQSLFLLLLWVPGSRG, SEQ ID NO 104), and a stop codon were also included.

### Example 2

### Generation of HBV Polyepitope MVA Vector

A recombinant modified vaccinia virus Ankara (MVA), expressing the same HBV polyepitope protein as present in INX102-3697, was generated by homologous recombination into deletion III of MVATGN33 (Transgene, France) using a shuttle plasmid containing the pDNA HBV vaccine gene construct functionally linked to the vaccinia virus H5R early late promoter. The MVA vector (INX102-0557), was amplified and produced on chicken embryonic fibroblasts, purified by a multi-step low speed centrifugation process and resuspended in 10 mM Tris-HCl, 5% (w/v) saccharose, 10 mM sodium glutamate, 50 mM NaCl, pH 8.0 at an infectious titer of 3E+08 pfu/ml.

### Example 3

### Generation of a Polyepitope Protein

Generation of Recombinant E. coli Strains

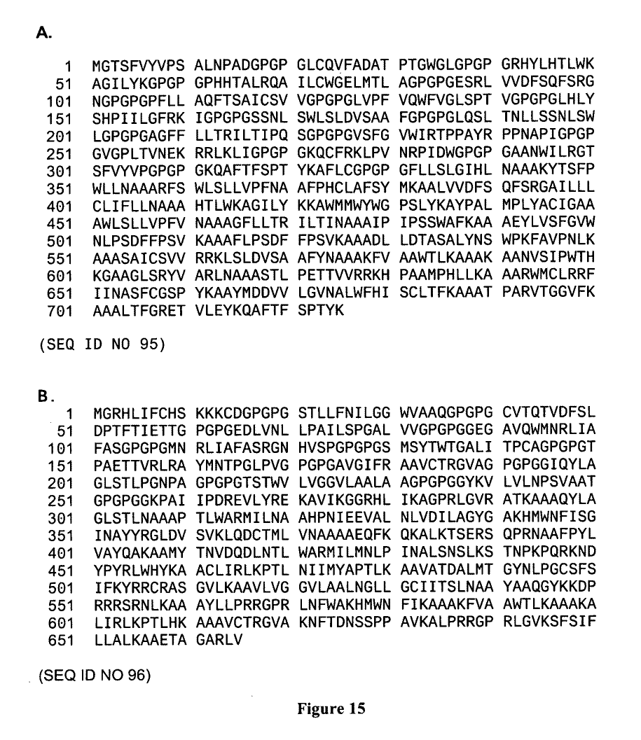

Based on the amino acid sequence of the HBV polyepitope protein (FIG. 15A—SEQ ID 95) an optimized coding sequence was designed and synthesized by GeneArt (Regensburg, Germany) using their GeneOptimizer sequence optimization software. During design, appropriate endonuclease restriction sites were introduced in the 5′ and 3′ flanking regions to simplify subcloning into the expression vectors, and a metal-affinity tag (tag represented by the amino acid sequence HHHMFHHHWWHHHMWHHH (LHH11), SEQ ID NO 97; or a hexahistidine tag (His-6), SEQ ID NO105) was added preceded by a two amino acid (NA) linker sequence. In case of His-6, the tag was preceded by a small linker sequence of 5 amino acids (PGSLE, SEQ ID NO 106) for subcloning purposes.

Based on the amino acid sequence of the HCV polyepitope protein (FIG. 15B—SEQ ID 96) an optimized coding sequence was designed and synthesized by GeneArt (Regensburg, Germany) using their GeneOptimizer sequence optimization software. During design, appropriate endonuclease restriction sites were introduced in the 5′ and 3′ flanking regions to simplify subcloning into the expression vectors, and an affinity tag (represented by SEQ ID NO 97; LHH11) was added preceded by a three amino acid (NAA) linker sequence.

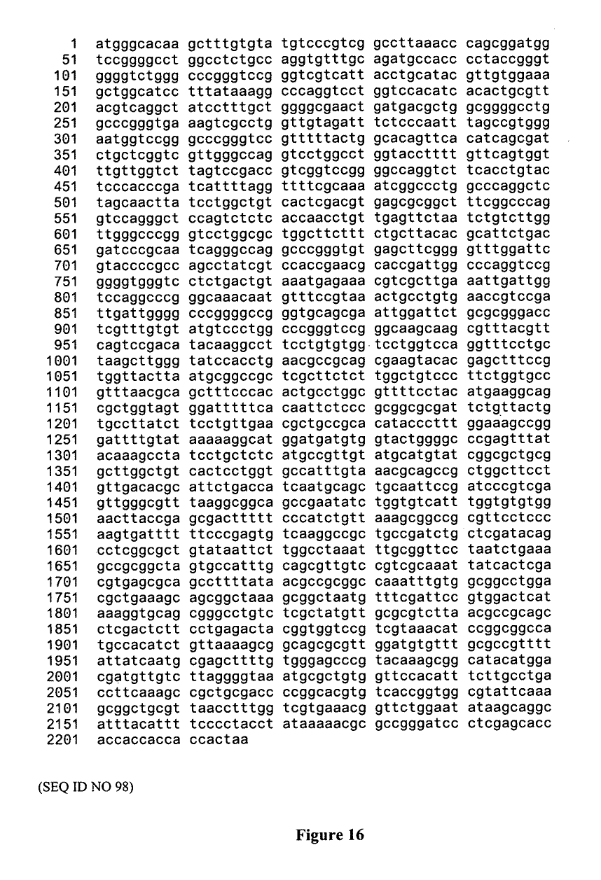

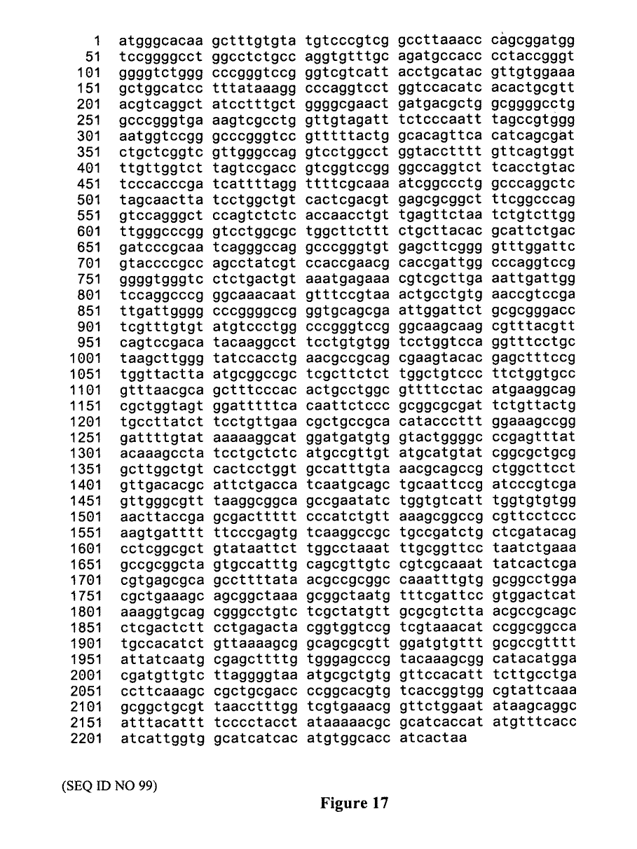

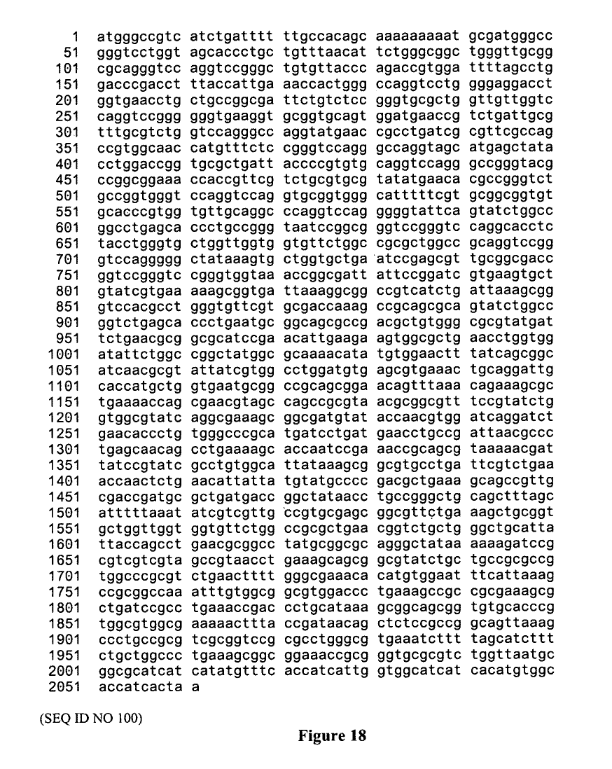

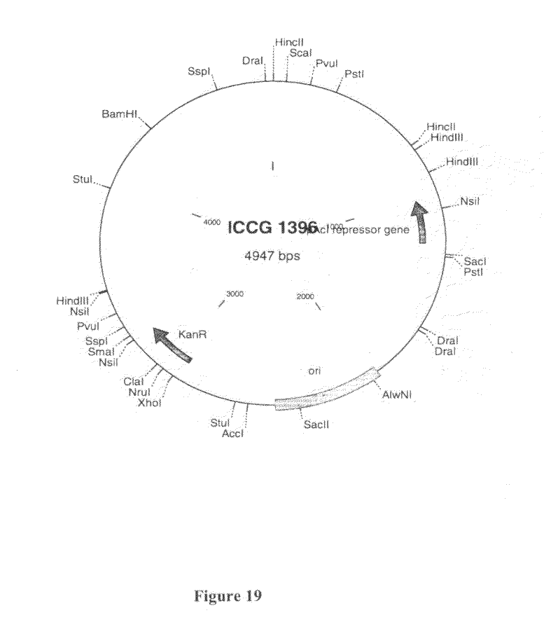

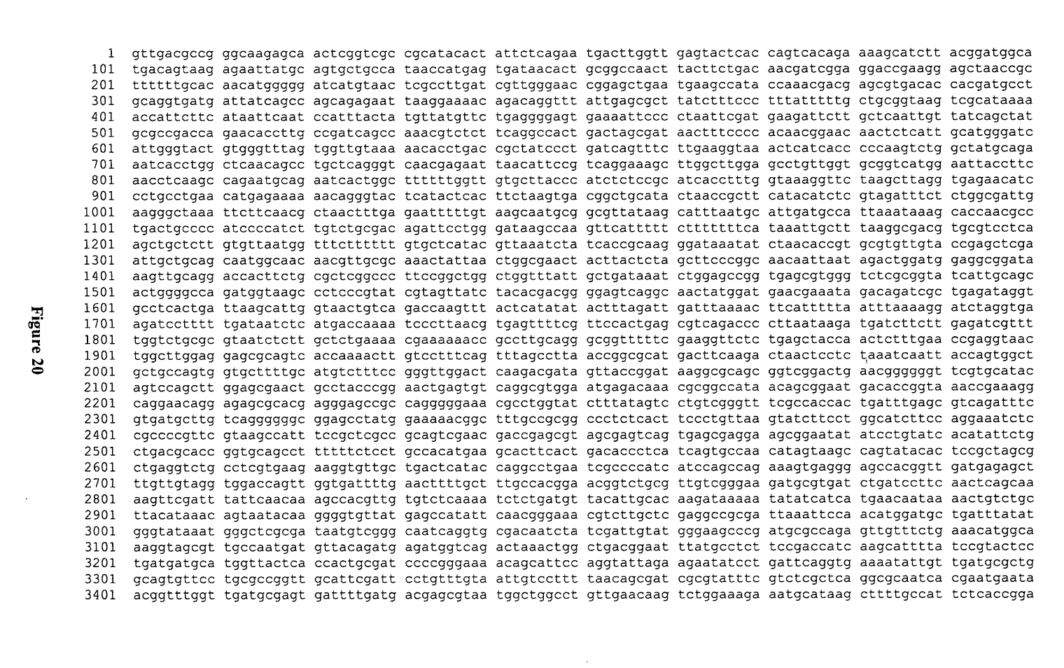

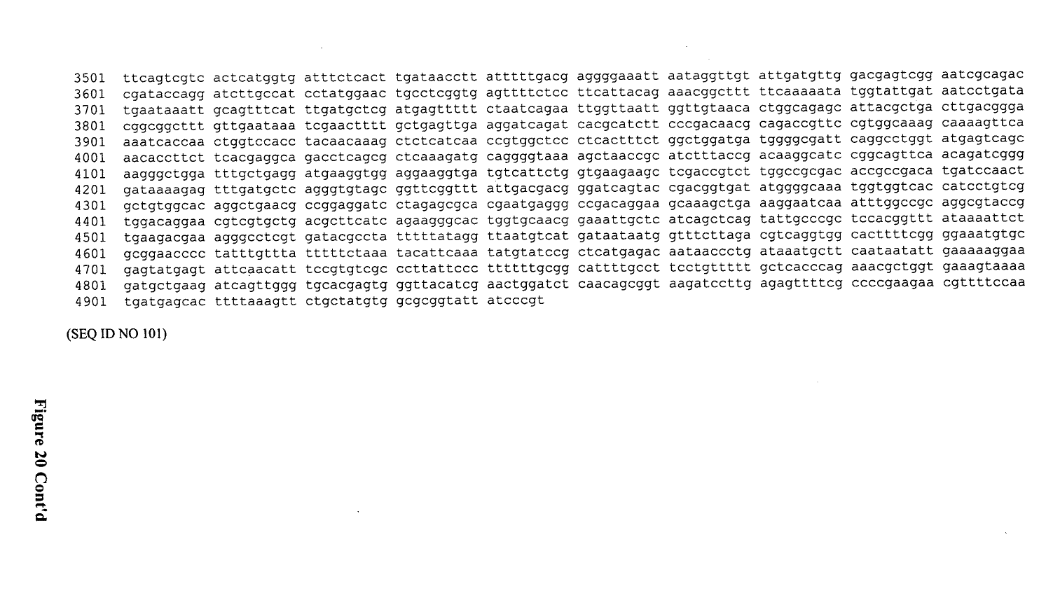

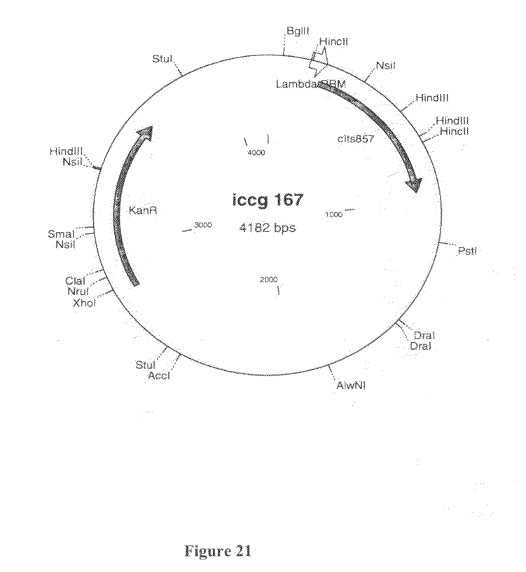

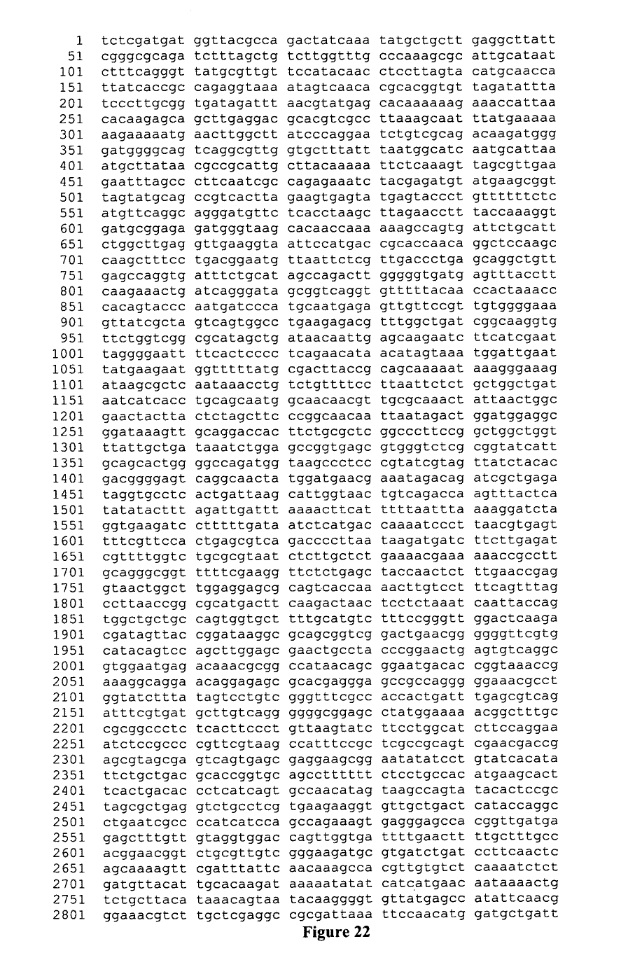

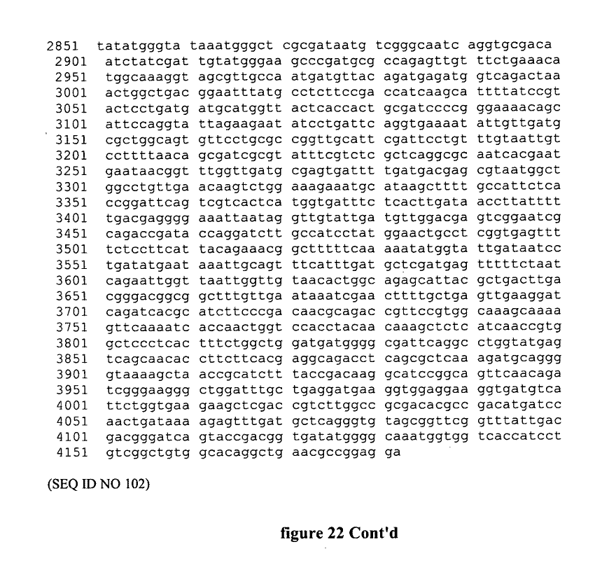

The complete HBV and HCV polyepitope coding regions (FIGS. 16, 17, 18—SEQ ID NO 98, SEQ ID NO 99, and SEQ ID NO 100 respectively) were subcloned into E. coli vectors for expression using the temperature-inducible bacteriophage Lambda pR-based expression system known in the art. The final expression plasmids were transformed by a standard heat-shock method into competent E. coli host strains BL21 (Novagen, USA) and SG4044 (Gottesman et al., 1981) already transformed with resp. the plasmid pAcI (FIGS. 19-20) or plasmid pcI857 (FIGS. 21-22) ensuring the expression of the temperature-sensitive mutant of the bacteriophage Lambda cI repressor.

All subcloning was performed using standard recombinant DNA technology mainly based on the use of restriction enzymes and PCR techniques known in the art.

After transformation, individual colonies were transferred into culture medium consisting of 20 g/l of yeast extract (Becton Dickinson, ref. 212750 500G), 10 g/L of tryptone (Becton Dickinson, ref. 211705 500G), 5 g/L of NaCl and 10 mg/L of tetracycline, grown at 28° C. and induced by a temperature shift to 37° C. and/or 42° C. At several time intervals up to 4 hour post induction, samples (total cell lysates) of non-induced, induced, and wild-type cells were analyzed by western blot analysis with polyclonal rabbit antisera against the HBV and HCV polyepitope protein.

Production of HBV and HCV Polyepitope Proteins in E. coli (Fermentation)

The HBV and HCV polyepitope proteins were produced from a (pre)culture in medium consisting of 20 g/l of yeast extract (Becton Dickinson, ref 212750 500G), 10 g/L of tryptone (Becton Dickinson, ref. 211705 500G), 5 g/L of NaCl and 10 mg/L of tetracycline.

Preculture medium (500 mL in 2 L baffled shake flasks) was inoculated with 500 μL from a cell bank glycerol slant. Precultures were incubated at 28° C. and 200-250 rpm for 22 to 24 h. Baffled shake flasks (2 L) were filled with 500 mL of culture medium and inoculated 1/20 (v/v) with preculture broth. The culture was allowed to grow for 4 h at 28° C. and was induced for 3 h at 37° C. Cells were recovered from the culture broth by centrifugation in a Beckman MA10.500 rotor at 9000 rpm at 4° C. for 25 min. Cell pellets were stored at −70° C.

Purification of HBV Polyepitope Proteins Expressed in E. coli

The HBV polyepitope proteins were purified under denaturing conditions after cell disruption and inclusion body harvest/extraction. Since the HBV polyepitope proteins were expressed as metal affinity tagged proteins, capture and intermediate purification of the Gu.HCl-solubilized product could be performed on Ni²⁺-IMAC after disulphide bridge disruption, reversible cystein blocking and clarification.

In brief, cell pellets were resuspended in 5 volumes (5 mL buffer/gram wet weight cell pellet) of lysis buffer (50 mM Tris/HCl buffer, pH 8.0, to which 2 mM MgCl₂, 1/25 Complete from 25× stock solution and 10 U/mL benzonase purity grade II was added). After resuspension, cell disruption was performed by high-pressure homogenisation (1 pass at 1.4 kbar and 4° C.). Cell lysate obtained was subjected to centrifugation (18.500 g for 1 hour at 4° C.) and pellet fraction was recovered. The pellet was resuspended in 3 volumes (3 mL buffer/gram wet weight original cell pellet) of inclusion body wash buffer I (0.25 M Gu.HCl, 10 mM EDTA, 10 mM DTT, 1% sodium N-lauroylsarcosinate, 50 mM Tris/HCl pH 8.0) and stirred for 45 minutes at 20° C., followed by centrifugation (18.500 g for 30 minutes at 4° C.). After discarding the supernatans, the pellet was collected and resuspended in 1.5 volumes (1.5 mL buffer/gram wet weight original cell pellet) of inclusion body wash buffer II (10 mM EDTA, 10 mM DTT, 1% Triton X-100, 50 mM Tris/HCl pH 8.0). After stirring for 30 minutes at 20° C., suspension was centrifuged (18.500 g for 30 minutes at 4° C.). Next, pellet fraction obtained was subjected to a third inclusion body wash step by resuspension in 1.5 volumes (1.5 mL buffer/gram wet weight original cell pellet) of inclusion body wash buffer III (50 mM Tris/HCl buffer, pH 8.0, to which 2 mM MgCl₂, 1% Triton X-100 and 10 U/mL benzonase purity grade II was added) followed by stirring for 30 minutes at 20° C. and subsequent centrifugation at 18.500 g for 30 minutes (4° C.).

After recovery of the inclusion body (IB) pellet, HBV polyepitope protein was extracted by resuspending the IB-pellet in 9 mL extraction buffer (6.7M Gu.HCl, 56 mM Na₂HP0₄.2H₂0, pH 7.2) per gram inclusion body pellet (wet weight) and subsequent stirring for 60 minutes at 20° C. Then, the protein solution was clarified by centrifugation (18.500 g for 30 minutes at 4° C.). Soluble HBV polyepitope protein in the supernatant was sulfonated by addition of sodium sulfite, sodium tetrathionate and L-cystein to final concentrations of respectively 320 mM, 65 mM and 0.2 mM. After subsequent pH adjustment to pH 7.2, protein solution was stirred overnight at room temperature in contact with air and shielded from the light. Then, n-dodecyl-N,N-dimethylglycine (also known as lauryldimethylbetaine or Empigen BB®, Albright & Wilson) and imidazole were added to the protein solution to a final concentration of 3% (w/v) and 20 mM respectively and the pH was adjusted to pH 7.2.

All further chromatographic steps were executed on an Äkta Explorer 100 workstation (GE healthcare Bio-Sciences). The sample was filtrated through a 0.22 μm pore size membrane and loaded on a Ni²⁺-IDA column (Chelating Sepharose FF, GE healthcare Bio-Sciences), that was equilibrated with 50 mM phosphate, 6 M Gu.HCl, 3% Empigen BB®, pH 7.2 (IMAC-A buffer) supplemented with 20 mM imidazole. The column was washed sequentially with IMAC-A buffer containing 20 mM and 50 mM imidazole respectively till the absorbance at 280 nm reached the baseline level. Further washing and elution of the metal affinity tagged proteins was performed by the sequential application of IMAC-B buffer (50 mM phosphate, 6 M Gu.HCl, pH 7.2) supplemented with 50 mM imidazole, 200 mM imidazole and 700 mM imidazole respectively. The fractions were analyzed by SDS-PAGE (and silver staining) and western-blot using an antiserum directed against the HBV polyepitope protein.

Fractions containing the HBV polyepitope protein with >85% purity were pooled (‘IMAC pool’) and concentrated to approximately 3 mg protein/mL by ultrafiltration on a concentrator with MWCO 10 kDa (Centricon, Amicon, Millipore) that was previously blocked with PBS, 0.1% Tween 20 (incubation for 30 minutes at 37° C.) and subsequently thoroughly rinsed with IMAC-B buffer.

Final buffer exchange was performed by application of the concentrated ‘IMAC pool’ on a Sephadex G-25 (GE healthcare Bio-Sciences) desalting column that was equilibrated with 7M urea, 10% sucrose, 20 mM Tris/HCl, pH 8.0. Analysis of the main peak fractions by SDS-PAGE under non-reducing conditions (and Coomassie brilliant blue staining) and western-blot analysis using an antiserum directed against the HBV polyepitope protein confirmed product integrity and a purity of >85%.

Purification of HCV Polyepitope Proteins Expressed in E. coli

Ni²⁺-IMAC capture and intermediate purification performance was evaluated for the HCV polyepitope protein under denaturing conditions, after cell disruption by Gu.HCl-solubilization and disulphide bridge disruption, reversible cystein blocking and clarification.

In brief, cell pellet obtained from 2.7 L culture was resuspended in 10 volumes (10 mL buffer/gram wet weight cell pellet) of lysis buffer (6M Gu.HCl, 50 mM Na₂HP0₄.2H₂0, pH 7.2) and sodium sulfite, sodium tetrathionate and L-cystein were added to final concentrations of respectively 320 mM, 65 mM and 0.2 mM. After subsequent pH adjustment to pH 7.2, solution was stirred overnight at room temperature in contact with air and shielded from the light. The cell lysate obtained was clarified by centrifugation (18.500 g for 60 minutes at 4° C.). Pellet was discarded and the supernatant, containing the soluble fusion protein fraction, was recovered. Then, n-dodecyl-N,N-dimethylglycine (also known as lauryldimethylbetaine or Empigen BB®, Albright & Wilson) and imidazole were added to the protein solution to a final concentration of 3% (w/v) and 20 mM respectively and the pH was adjusted to pH 7.2. The sample was filtrated through a 0.22 μm pore size bottle top filter with prefilter (Millipore).

All further chromatographic steps were executed on an Äkta Explorer 100 workstation (GE healthcare Bio-Sciences). A XK 16/20 column (GE healthcare Bio-Sciences) was packed with 20 mL of Ni²⁺-charged Chelating Sepharose FF resin (GE healthcare Bio-Sciences) and equilibrated with 50 mM phosphate, 6 M Gu.HCl, 20 mM imidazole , pH 7.2 (IMAC-E buffer) supplemented with 3% Empigen BB®.

Next, the protein sample was loaded on the column. The column was washed sequentially with IMAC-E buffer containing 3% Empigen BB® and IMAC-E buffer without 3% Empigen BB® till the absorbance at 280 nm reached the baseline level. Further washing and elution of the fusion product was performed by the sequential application of IMAC-F buffer (20 mM Tris, 8 M urea, pH 7.2) supplemented with 20 mM imidazole, 50 mM imidazole, 200 mM imidazole and 700 mM imidazole respectively till the absorbance at 280 nm reached the baseline level.

All protein fractions obtained were analyzed by SDS-PAGE analysis under non-reducing conditions (+subsequent silver staining) and western-blotting using for specific detection, polyclonal rabbit antisera directed against the HCV fusion protein that were pré-incubated with E. coli lysate (MC 1061 (pAcI)+BL21 (pAcI)).

Protein concentration in the 200 mM and 700 mM imidazole IMAC elution pools was determined by measuring absorbance at 280 nm and subtraction of the absorbance at 320 nm, assuming that a protein solution of 1 mg/mL in a cuvette with 1 cm optical pathlength yields an absorbance at 280 nm of 1.5.

The protein was mainly recovered in the 700 mM imidazole fraction with >90% purity.

### Example 4

### Materials and Methods for Immunization and Read-Out

Mice: The derivation and characterization of the human HLA-A2/Kb transgenic (tg) mice was described previously (Vitiello et al., 1991). For all experiments homozygous HLA-A2/Kb tg were crossed with BALB/c mice. H-2^{b×d} mice (Balb/c×C57BL/6) can be used in a limited fashion to evaluate the immunogenicity of epitopes restricted to human HLA DR alleles as there is a significant degree of overlap in the binding motifs of HLA-DR and murine Class II molecules (Livingston et al., 2002). This F1 offspring is further referred to as HLA-A02.1×BALB/c F1.

Cell lines and peptide loading: Jurkat cells stably transfected with HLA-A02 (2×10E4 cells/well) were used in CTL assays as antigen presenting cells (APC). The APC expressed the same chimeric molecule composed of the α1 and α2 domain of HLA Class I and the α3 domain of murine Class I antigens identical to the transgenic mice. The cells are loaded for 2 hours at a density of 10⁶ cells/ml with 10 μg/ml of peptide (Table A). Cells were washed once to remove the excess of peptide and then added to the CD8+ spleen cells.

TABLE A
HLA-A2-restricted HBV peptides

| Epitope | IGP number | Sequence | SEQ ID NO |
|---|---|---|---|
| Env 335 | 2530 | WLSLLVPFV | 10 |
| Env 183 | 2531 | FLLTRILTI | 11 |
| Core 18 | 2287 | FLPSDFFPSV | 15 |
| Pol 455 | 2541 | GLSRYVARL | 21 |
| Pol 562 | 2521 | FLLSLGIHL | 1 |
| Pol 538 | 2546 | YMDDVVLGV | 26 |

For the evaluation of HLA Class II epitopes, irradiated syngeneic spleen cells (2×10E5 cells/well) were used as APC in vitro. These APC were loaded for 2 to 4 hours at a density of 5×10⁶ cells/ml with 10 μg/ml of peptide (Table B). Cells were γ-irradiated at 10 Gy, washed and pipetted through a cell strainer before addition to the wells

TABLE B
DR-restricted HBV peptides

| | IGP | | |
|---|---|---|---|
| Epitope | number | Sequence | SEQ ID NO |
| pol 774 | 2619 | GTSFVYVPSALNPAD | 31 |
| pol 420 | 2627 | SSNLSWLSLDVSAAF | 39 |
| pol 501 | 2628 | LHLYSHPIILGFRKI | 38 |
| pol 523 | 2629 | PFLLAQFTSAICSVV | 36 |
| PADRE | 2465 | AKFVAAWTLKAAA | 47 |

Preparation of CD4+ and CD8+ cells: From each individual mouse, the spleen was removed and spleen cells were isolated in pools of three mice. CD8+ or CD4+ cells were purified by magnetic separation using anti-CD8 or anti-CD4 antibody-coated magnetic MACS beads (Miltenyi), according to the manufacturer's instructions.

Interferon-gamma ELISPOT: A 96-well ELISPOT plate (MAIP HTS plates, Millipore) was coated overnight with an anti-IFN-γ antibody (clone AN18, MabTech) and blocked for 2 hours at room temperature with RPMI-1640 medium supplemented with 5% Fetal Bovine serum. In triplicate, CD8+ (5×10E4 and 2×10E5 cells/well) or CD4+ spleen cells (2×10E5 cells/well) together with peptide-loaded antigen presenting cells were added. Plates were then left undisturbed overnight. After incubation, cells were removed, plates were washed several times and incubated with biotinylated anti-IFN-γ antibody (clone R46A2, MabTech) for 2 hours at room temperature. After washing, plates were incubated with streptavidin-HRP (BD Biosciences) for 1 hour at room temperature. Then, spots were visualized using AEC (BD Biosciences) as substrate. Rinsing the plates with tap water stops the color reaction. Plates are then dried and analyzed using an'automated ELISPOT reader (AELVIS).

Responses were reported as spot forming cells (SFC)/10⁶ cells and the criteria for determining the immunogenicity for individual epitopes were established using background responses measured with unloaded antigen presenting cells as negative control condition. Any delta response ≧30 SFC/10⁶ cells with a response ratio ≧2 was classified as positive. To quantify CTL responses either the values from plates with 5×10E4 or 2×10E5 CD8+ cells/well were used based on the possibility to read the number of spots correctly (i.e.: very strong responses were measured using the 5×10E4 CD8+ cells/well plated and lower responses were measured using the 2×10E5 CD8+ cells/well plates).

Interleukin-5 ELISPOT: A 96-well ELISPOT plate (MAIP HTS plates, Millipore) was coated overnight with an anti-IL-5 antibody (clone TRFK5, Mabtech) and blocked for 2 hours at room temperature with RPMI-1640 medium supplemented with 5% Fetal Bovine serum. In triplicate, CD4+ spleen cells (2×10E5 cells/well) together with peptide-loaded antigen presenting cells were added. Plates were then left undisturbed overnight. After incubation, cells were removed, plates were washed several times and incubated with biotinylated anti-IL-5 antibody (clone TRFK4, Mabtech) for 2 hours at room temperature. After washing, plates were incubated with streptavidin-HRP (BD Biosciences) for 1 hour at room temperature. Then, spots were visualized using AEC (BD Biosciences) as substrate. Rinsing the plates with tap water stops the color reaction. Plates are then dried and analyzed using an automated ELISPOT reader (AELVIS).

Responses were reported as spot forming cells (SFC)/10⁶ cells and the criteria for determining the immunogenicity for individual epitopes were established using background responses measured with unloaded antigen presenting cells as negative control condition. Any delta response ≧30 SFC/10⁶ cells with a response ratio ≧2 were classified as positive.

### Example 5

### Use of Adjuvanted HBV Polyepitope Protein as Prime Before MVA Boost

Preparation of Immunogens

Formulation with aluminium: the HBV-His6 polyepitope protein (E. coli BL21 derived, 926 μg/ml in 20 mM Tris, 7M Urea, 10% sucrose) was formulated with Aluminum hydroxide (Alhydrogel, Brenntag biosector) by adding 1 volume of protein to 1 volume of Alhydrogel (1.3%). The suspension was shaken for 3 hours at room temperature and further incubated at 4° C. for about 20 hours.

Formulation with RIBI: equal volumes of protein and RIBI (MPL®+TDM Emulsion (Sigma, M6536)) (1 vial emulsified in 1 ml PBS after 5′ pre-warming at 45° C.) were vortexed (3′, speed 1800 rpm) just before injection.

The pvp-DNA was diluted ½ with PBS.

Immunization Schedule

Two groups of 18 HLA-A2/Kb tg×BALB/c F1 mice received 3-subcutaneous injections of 100 μg of the HBV polyepitope protein formulated in RIBI or Alhydrogel with a two-week interval (homologous prime-boost immunization). Two groups of 18 mice were given two subcutaneous injections with a two-week interval of 100 μg of the HBV polyepitope protein formulated in either RIBI or Alhydrogel, followed by a heterologous subcutaneous boost injection with 10⁷ pfu HBV MVA three weeks later. As a reference immunization, 18 mice were immunized with a HBV DNA prime injection of 100 μg (intramuscularly in the m. tibialis anterior divided over two subsequent immunizations with a three-day interval), followed 3 weeks later by a HBV MVA boost injection of 10⁷ pfu. The timing of the immunizations in the different groups was adapted to allow read out in all groups at the same time, 11 to 13 days after the last immunization.

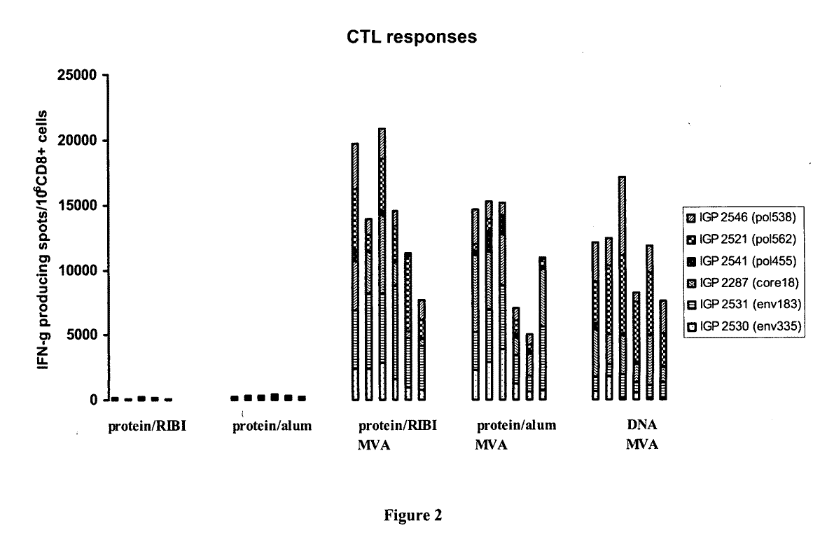

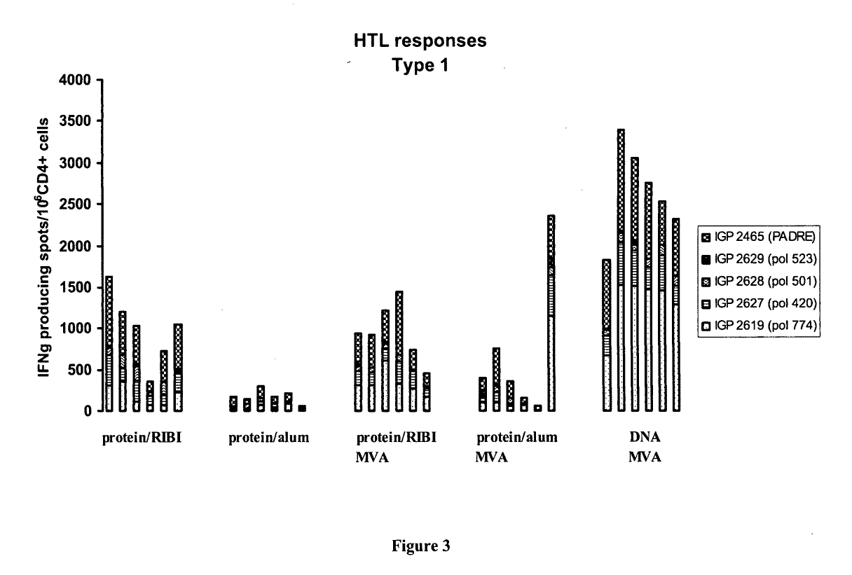

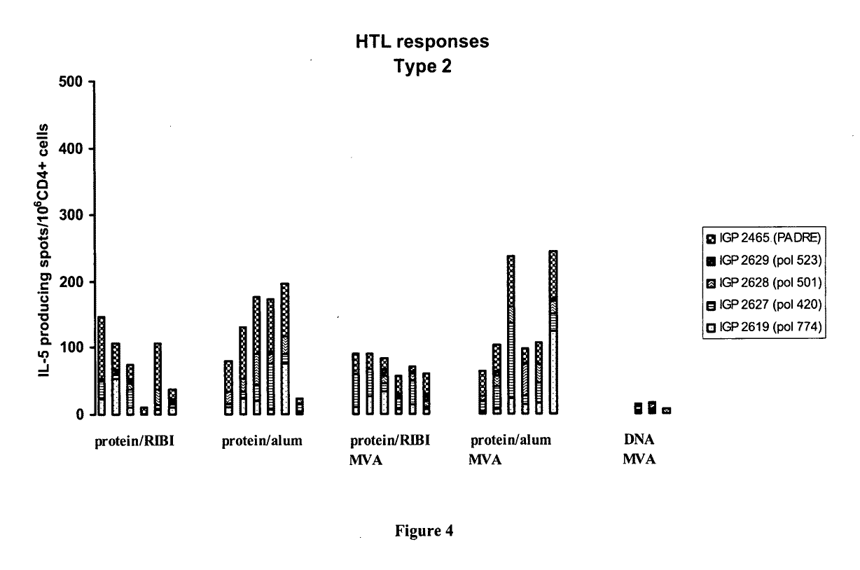

Results (FIGS. 2-4)

Immunization with the HBV-His6 protein without MVA boost elicited only very low HBV-specific HLA-A02.1-restricted CTL responses. HLA-DR restricted HTL type 1 and HTL type 2 responses were more readily detectable and clearly show the Th2 skewing by Alhydrogel formulation opposite to the Th1 skewing by RIBI formulation.

The heterologous boost immunization with MVA spectacularly increased CTL responses compared to a homologous prime-boost immunization with protein only. This heterologous prime-boost immunization elicited similar responses as compared to the reference immunization using pvp-formulated HBV DNA followed by a single MVA boost injection. The use of two different adjuvantia (RIBI and Alhydrogel) did not influence the height of the obtained CTL responses. This is surprising and unexpected in view of the different Th1/Th2 skewing observed for the different regimens. More particularly, the Alhydrogel primed responses were more skewed towards Th2, than the RIBI primed responses. The DNA priming/MVA boosting gave an almost exclusive Th1 type response; nevertheless and unexpectedly the CTL responses obtained with protein formulated with Alhydrogel and an MVA boost were at least as high.

### Example 6

### Use of Unadjuvanted HBV Polyepitope Protein as Prime Before MVA Boost

Preparation of Immunogens

Formulation with aluminium: the HBV-His6 polyepitope protein (E. coli BL21 derived, 1.10 mg/ml in 20 mM Tris, 7M Urea, 10% sucrose) and HBV-His6 polyepitope protein (E. coli SG4044 derived, 1.15 mg/ml in 20 mM Tris, 7M Urea, 10% sucrose) were diluted to 1 mg/ml in PBS. Formulation with Aluminum hydroxide (Alhydrogel, Brenntag biosector) was done by adding 1 volume of diluted protein to 1 volume of Alhydrogel (1.3%). The suspension was shaken for 3 hours at room temperature and further incubated at 4° C. for about 20 hours.

Formulation in buffer: HBV-His6 polyepitope protein (E. coli BL21 derived, 1.10 mg/ml in 20 mM Tris, 7M Urea, 10% sucrose) and HBV-His6 polyepitope protein (E. coli SG4044 derived, 1.15 mg/ml ml in 20 mM Tris, 7M Urea, 10% sucrose) were diluted to 1 mg/ml in PBS.

Immunization Schedule

Four groups of 12 HLA-A2/Kb tg×BALB/c F1 mice received 2 subcutaneous injections of 100 μg of the HBV polyepitope protein derived from BL21 or SG4044 and formulated in PBS or Alhydrogel with a two-week interval, followed by a heterologous subcutaneous boost injection with 10⁷ pfu HBV MVA three weeks later. A fifth group only received the MVA immunization without any protein prime. The timing of the immunizations in the different groups was adapted to allow read out in all groups at the same time, 11 to 13 days after the last immunization.

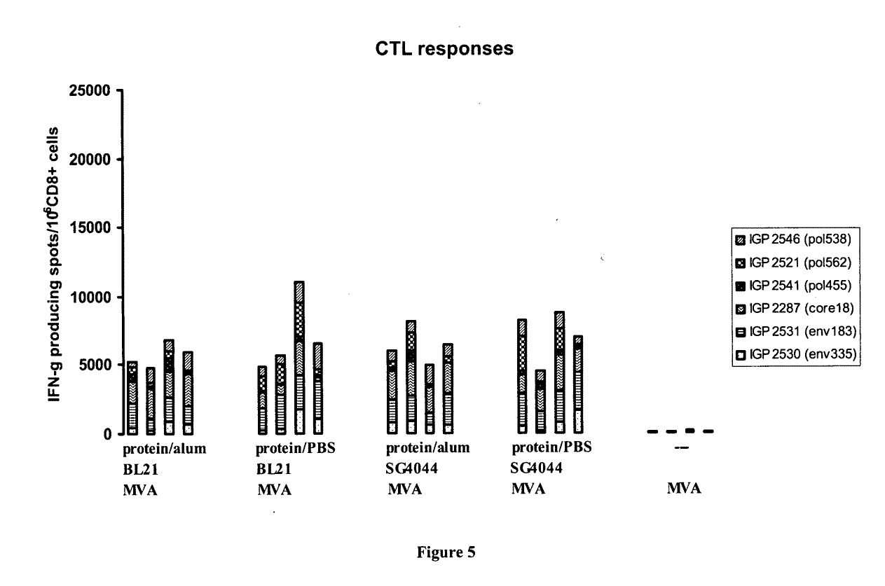

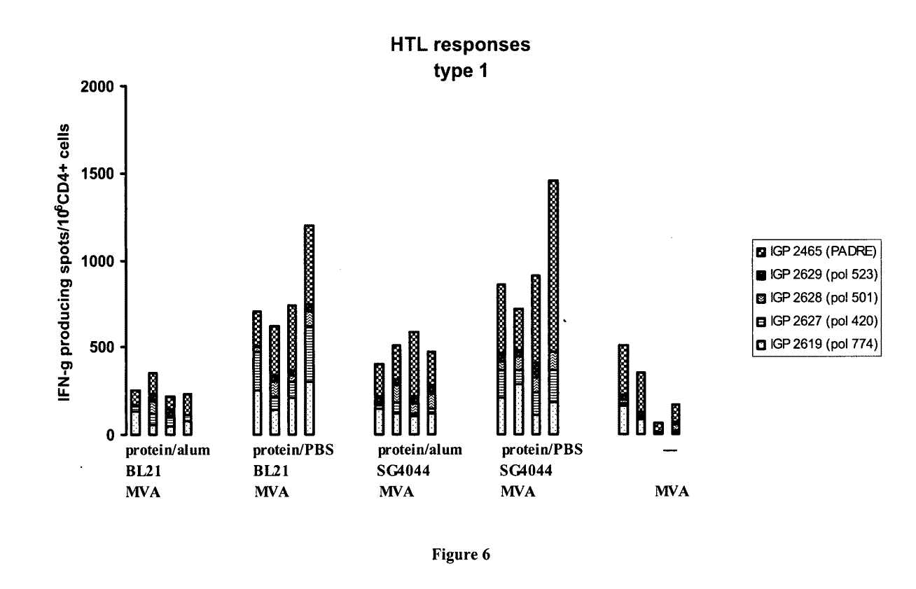

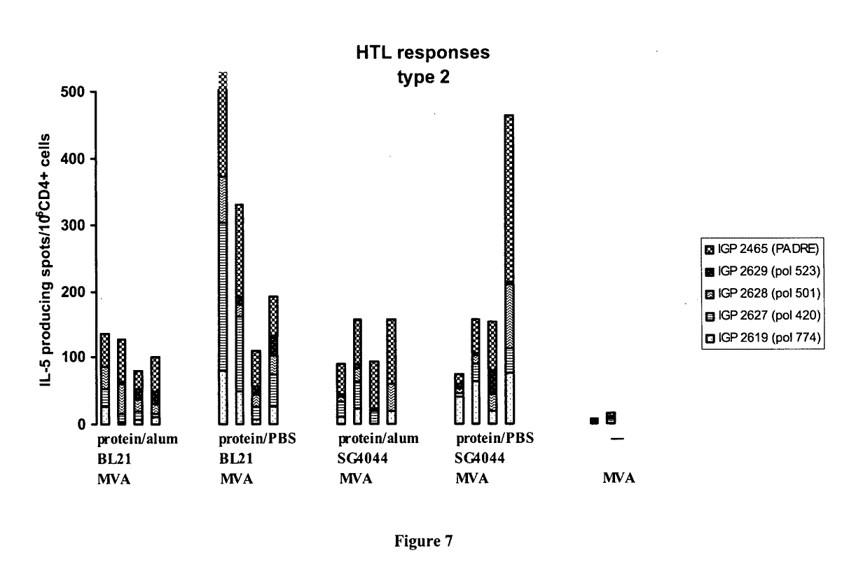

Results (FIGS. 5-7)

The heterologous boost immunization with HBV MVA again provided spectacularly high CTL responses. The use of two different formulations (PBS and Alhydrogel) or the source of the antigen (BL21 or SG4044) did not influence the level of the obtained CTL response. Although the Th1/Th2 balance was similar in all groups that receive the protein prime, it should be noted that Alhydrogel formulations had an overall lower Th1 and Th2 response. The MVA alone immunization induced only extremely weak CTL responses This experiment confirms the surprising effect of polyepitope proteins which can be used with Th2 inducing or even without any adjuvant to prime for CTL responses which can be significantly boosted by a heterologous immunization.

### Example 7

### Superiority of HBV Polyepitope Protein Prime/MVA Boost Over MVA Prime/HBV Polyepitope Protein Boost

Preparation of Immunogens

Formulation with aluminium: the HBV-His6 polyepitope protein (E. coli SG4044 derived, 1.14 mg/ml in 20 mM Tris, 7M Urea, 10% sucrose) was diluted to 1 mg/ml in PBS. Formulation with Aluminum hydroxide (Alhydrogel, Brenntag biosector) was done by adding 1 volume of diluted protein to 1 volume of Alhydrogel (1.3%). The suspension was shaken for 3 hours at room temperature and further incubated at 4° C. for about 20 hours.

Immunization Schedule

Four groups of 12 HLA-A2/Kb tg×BALB/c F1 mice received 2 subcutaneous injections of 100 μg of the HBV polyepitope protein formulated with Alhydrogel with a two-week interval, followed by a heterologous subcutaneous boost injection with 10⁷, 10⁶, 10⁵ or 10⁴ pfu HBV MVA three weeks later. A fifth group received 2 subcutaneous injections of 10⁷ pfu MVA with a two week interval, followed by s heterologous subcutaneous boost injection of 100 μg of the HBV polyepitope protein formulated with Alhydrogel three weeks later. The timing of the immunizations in the different groups was adapted to allow read out in all groups at the same time, 11 to 13 days after the last immunization.

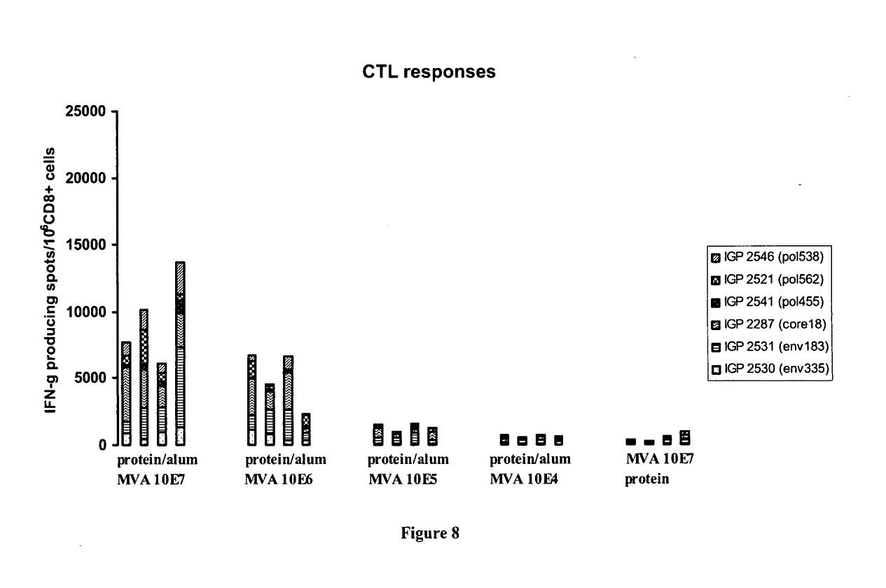

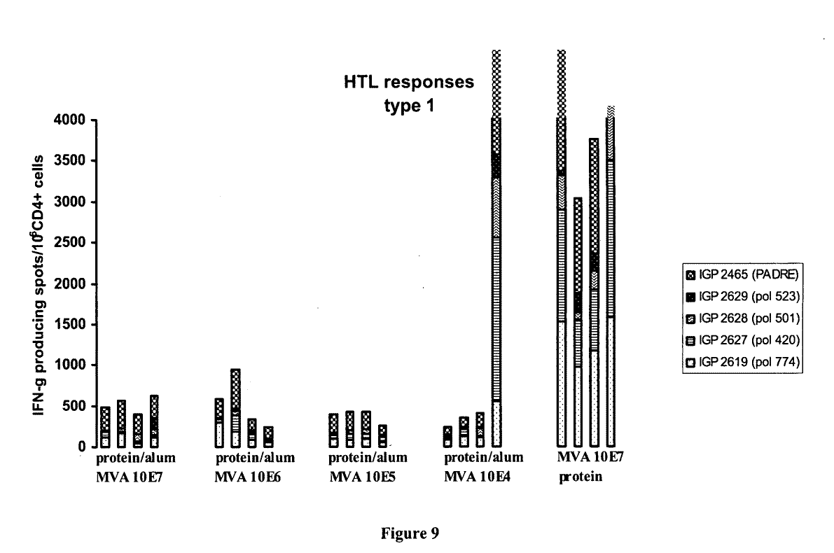

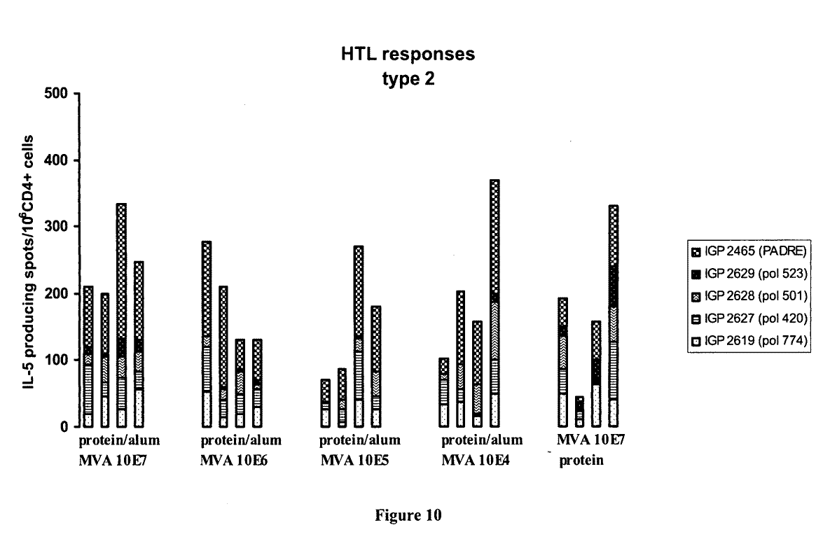

Results (FIGS. 8-10)

The heterologous boost immunization with HBV MVA again provided spectacularly high CTL responses. A clear dose response for the MVA boost is noted. The low dose of only 10⁴ pfu MVA boost was still equally potent to induce CTL responses as the 1000-fold higher dose of 10⁷ dose of MVA used for priming prior to protein boosting. This clearly shows that polyepitope protein priming followed by MVA boost is superior over the reverse order of administration and this in spite of a very strong HTL and more specifically Th1 response in the latter.

### Example 8

### Use of HBV Polyepitope Protein as Prime Before DNA Boost

Preparation of Immunogens

Formulation in buffer: the HBV-LHH11 polyepitope protein (E. coli BL21 derived, 2;43 mg/ml in 20 mM Tris, 7M Urea, 10% sucrose) was diluted with 20 mM Tris, 7M Urea, 10% sucrose to 1 mg/ml.

The pvp-DNA was diluted ½ with PBS.

Immunization Schedule

Two groups of 18 HLA-A2/Kb tg×BALB/c F1 mice received 2 subcutaneous injections of 100 μg of the HBV polyepitope protein with a two-week interval. The first of these groups received after 3 weeks a heterologous subcutaneous boost with 10⁶ pfu HBV MVA three weeks later. The second group received after 3 weeks a heterologous intramuscular boost with 100 μg HBV DNA three weeks later.

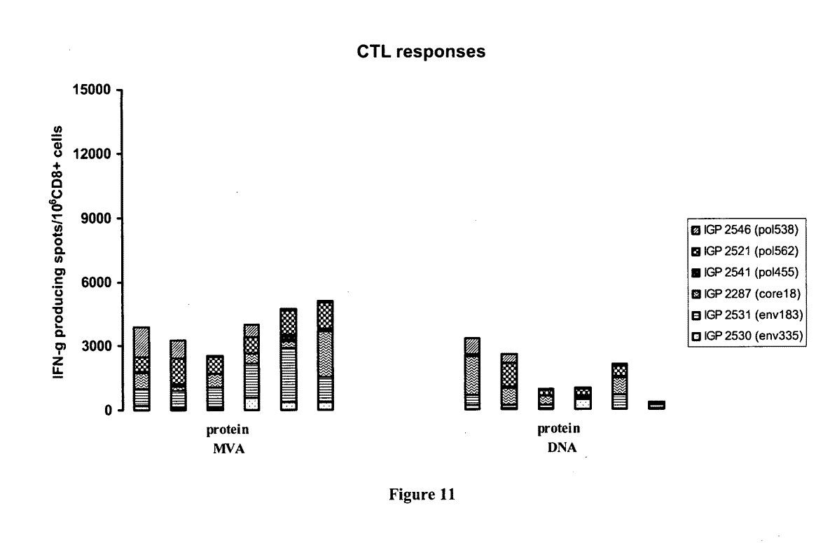

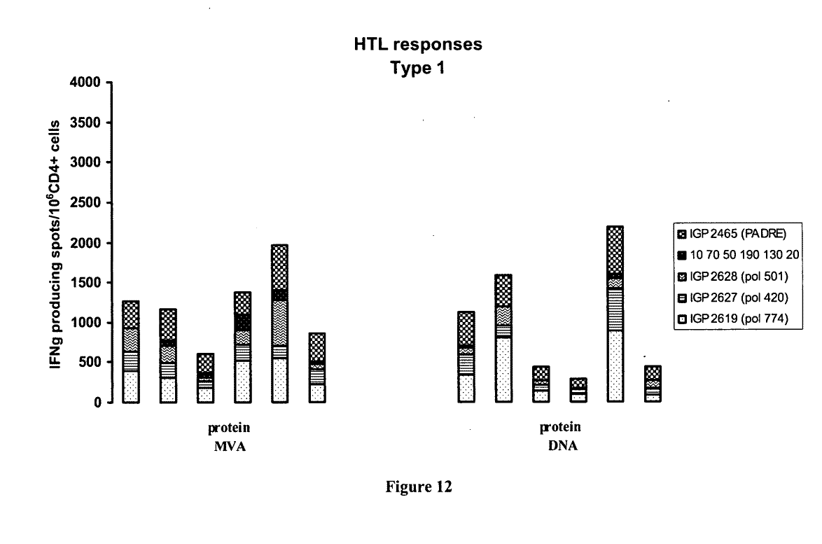

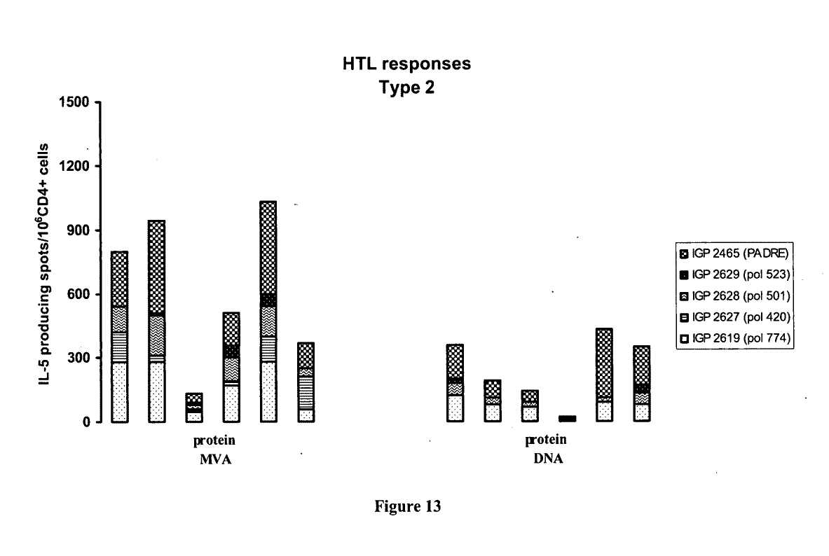

Results (FIGS. 11-13)

The use of DNA as an alternative to MVA in the boost resulted also in a significant boost effect. From example 5 it is known that even 3 injection of protein result only in very low CTL responses. Similarly it is known that a single injection with DNA without cardiotoxin pre-treatment only results in minimal CTL activity. This result is surprising as DNA is typically used as a priming composition and not as a booster. Apparently, in combination with protein this classical order can be reversed and responses primed by protein can also be boosted by DNA. Most remarkably, the polyepitope protein in this experiment was not adjuvanted. Based on the experience in example 6 it is logical to expect that also aluminium formulations would allow an efficient protein prime DNA boost in spite of the lack of Th1 skewing by the protein priming.

### Example 9

### Improved Immune Responses by Repeated Cycles of Polyepitope Protein/MVA Prime/Boost

Preparation of Immunogens

Formulation in buffer: the HBV-LHH11 polyepitope protein (E. coli BL21 derived, 2.43 mg/ml in 20 mM Tris, 7M Urea, 10% sucrose) was diluted with 20 mM Tris, 7M Urea, 10% sucrose to 1 mg/ml.

Immunization Schedule

Three groups of 18 HLA-A2/Kb tg×BALB/c F1 mice received 2 subcutaneous injections of 100 μg of the HBV polyepitope protein with a two-week interval, followed by a heterologous subcutaneous boost injection with 10⁶ pfu HBV MVA three weeks later (first cycle). A first group received no further injections (this group is referred to as PPM).The second group received after 3 weeks a second cycle consisting of 2 subcutaneous injections of 100 μg of the HBV polyepitope protein with a two-week interval (this group is referred to as PPMPP). The third group received after three weeks a second cycle consisting of 2 subcutaneous injections of 100 μg of the HBV polyepitope protein with a two-week interval, followed by a heterologous subcutaneous boost injection with 10⁶ pfu HBV MVA three weeks later (this group is referred to as PPMPPM). The timing of the immunizations in the different groups was adapted to allow read out in all groups at the same time, 11 to 13 days after the last immunization.

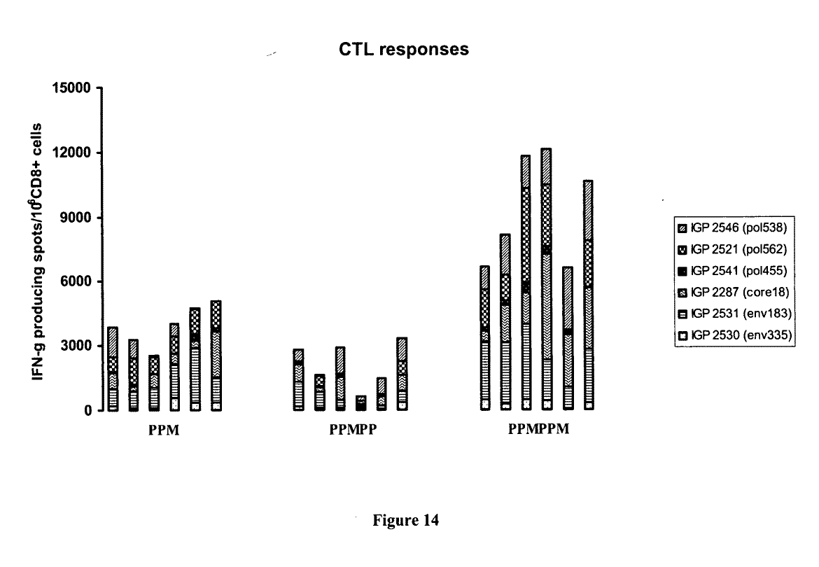

Results (FIG. 14)

The use of a complete second cycle of protein/MVA resulted in much higher CTL responses. This effect is not noted after the partial second cycle consisting of protein only. In this case CTL responses are even lower than after a single cycle of protein/MVA.

### Example 10

### Use of HCV Polyepitope Protein as Prime Before DNA Boost in Different HLA Transgenic Mice

Preparation of Immunogens

The purified HCV protein as obtained in example 3 was diluted in desalting buffer (7M Urea, 20 mM Tris, 10% sucrose, pH 8) towards a concentration of 1 mg/ml and 100 μl (100 μg) was injected subcutaneously at the base of the tail using a BD Microfine™ plus 1.0 cc insulin syringe.

The pMB75.6 vector comprising the nucleotide sequence represented by SEQ ID NO 100, generated as described in example 3, was diluted with PBS towards a concentration of 1 mg/ml and 100 μg was administered by bilateral injection of 50 μl in both m. tibialis anterior (after anaesthesia).

Immunization Schedule

Two groups of 18 homozygous HLA-A24/K^{b} and one group of 18 homozygous HLA-A11/K^{b} transgenic mice were included. One group of each received a double protein prime and DNA as boost. As a control, the second group of HLA-A24/K^{b} transgenic mice received only a single DNA injection (without prior cardiotoxin pre-treatment).

| | strain | Week 0 and week 2 | Week 5 |
|---|---|---|---|
| 1 | HLA-A24/K^{b} | 100 μg | 100 μg |
| | | (HTL-CTL)-LHH11_HCV | (CTL-HTL)_HCV |
| | | protein | DNA |
| 2 | HLA-A24/K^{b} | / | 100 μg |
| | | | (CTL-HTL)_HCV |
| | | | DNA |
| 3 | HLA-A11/K^{b} | 100 μg | 100 μg |
| | | (HTL-CTL)-LHH11_HCV | (CTL-HTL)_HCV |
| | | protein | DNA |

All injections with the polyepitope protein were administered subcutaneously at the base of the tail at a 100 μg dose. DNA injections were given intramuscularly in the m. tibialis anterior at a 100 μg dose.

Mice were euthanized 11 days after the last injection. ELISPOT analyses for CTL responses (using methods as described in example 4) were performed on pooled spleen cells from 3 mice within the same immunization group. ELISPOT analyses for Th1 (IFNγ) were performed on pooled spleen cells from all 18 mice.

Results

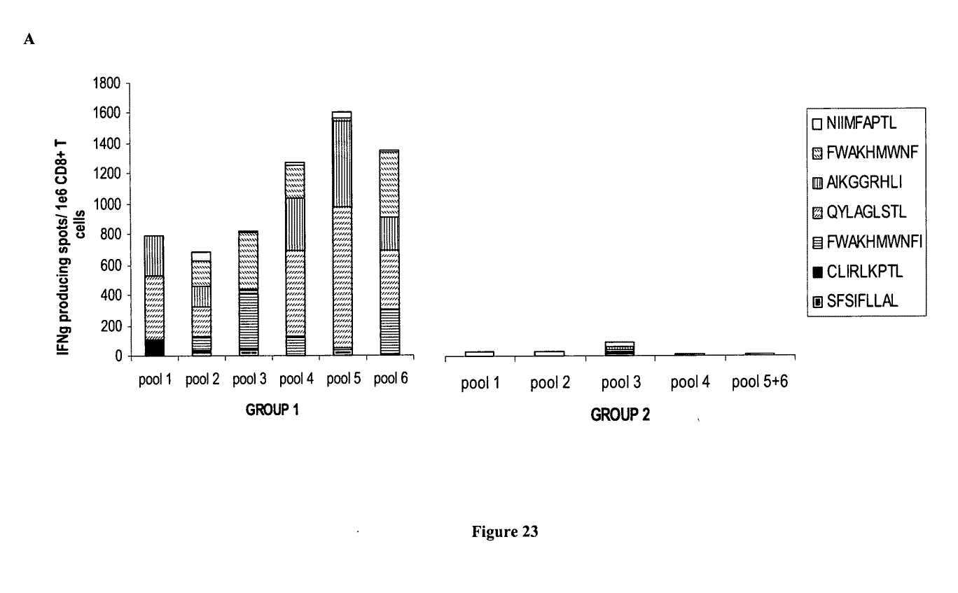

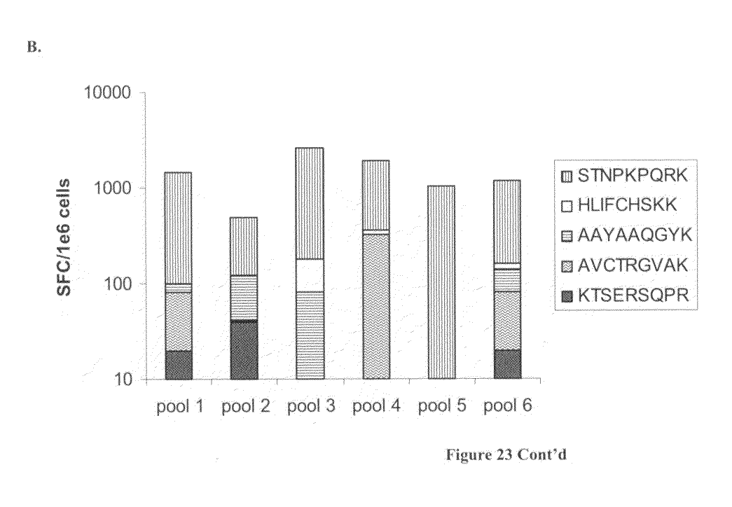

HCV-specific HLA-A24-restricted and HLA-A11-restricted CTL responses were highly increased when a protein prime immunization was boosted with DNA (FIGS. 23A and 23B). Positive responses towards both HLA-A24-restricted and to HLA-A11-restricted epitopes were detected. These responses were higher than the responses following DNA only immunizations as shown for the HLA A24-restricted epitopes.

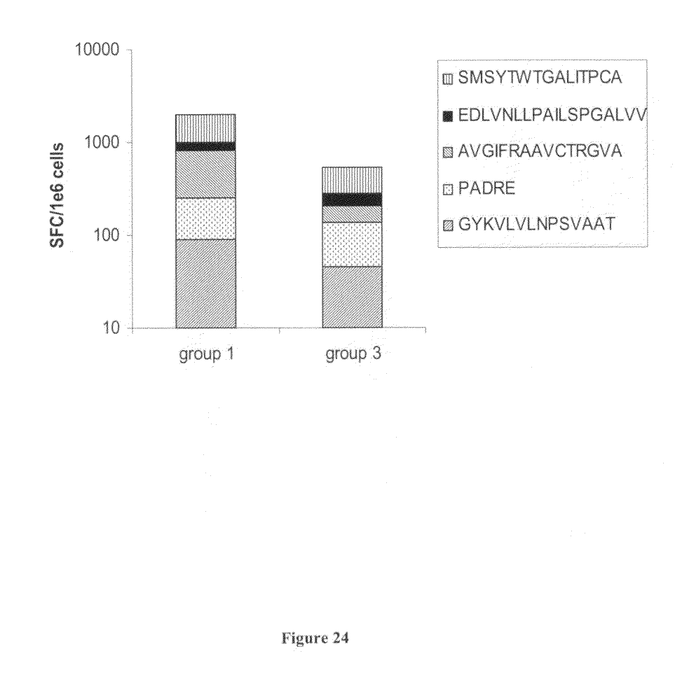

Also high T helper 1 responses could be detected upon protein priming, followed by DNA boosting (FIG. 24).

This demonstrates that the HCV polyepitope protein is especially useful as a priming agent in a heterologuous prime/boost treatment regimen.

### REFERENCES

Alexander et al. J Immunol. 2002, 168: 6189-6198.

Donnelly J J, Ulmer J B, Shiver J W, Liu M A. DNA vaccines. Annu Rev Immunol. 1997; 15:617-48.

Donnelly J J, Ulmer J B, Liu M A. DNA vaccines. Life Sci. 1997a; 60(3):163-72.

Feigner, et al., Proc. Nat'l Acad. Sci. USA 84:7413, 1987

Fernando G J, et al. J Immunol. 1998 Sep. 1; 161(5):2421-7.

Gottesman S. et al. (1981) Cell, 24(1), 225-233

Gupta R. K., et al. In: Vaccine Design and Adjuvant Approach, eds M. F. Powell and M. J. Newman. Plenum Press, NY, 1995; pp 229-248.

Livingston B., C. Crimi, M. Newman, Y. Higashimoto, E. Appella, J. Sidney, and A. Sette (2002) J. Immunol. 168:5499-5506.

HogenEsch H. Vaccine 2002; 20:S34-S39

Mannino & Gould-Fogerite, BioTechniques 6(7): 682, 1988

McConkey S J, et al. Nat Med. 2003 June; 9(6):729-35.

Mwau M, et al. J Gen Virol. 2004 April; 85(Pt 4):911-9.

Stover C K, et al. Nature. 1991 Jun. 6; 351(6326):456-60.

Thomson, S. A. et al., J Immunol. 157:822, 1996

Vitiello A., D. Marchesini, J. Furze, L. A. Sherman, R. W. Chesnut (1991) J. Exp. Med. 173:1007-1015.

Vuola J M, et al. J Immunol. 2005 Jan. 1; 174(1):449-55.

Wilson C. C., D. McKinney, M. Anders, S. MaWhinney, J. Forster, C. Crimi, S. Southwood, A. Sette, R. Chesnut, M. J. Newman, and B. D. Livingston (2003) J. Immunol. 171:5611-5623.

1. A method of priming a T cell response against at least one target antigen in a prime boost treatment regimen, comprising administering a polypeptide comprising a polyepitope construct comprising at least two CTL epitopes to a person in need of said priming in a prime boost treatment regimen, wherein the polypeptide is not linked to, combined with or included within a compound selected from the group consisting of: a particle formation promoting protein, a carrier protein and a CTL response inducing adjuvant.

2. The method according to claim 1, wherein the polyepitope construct comprises at least 10 isolated CTL epitopes.

3. The method according to claim 1, wherein the polyepitope construct further comprises one or more HTL epitopes.

4. The method according to claim 1, wherein two or more of the epitopes in the construct are linked by one or more spacer amino acids.

5. The method according to claim 1, wherein the at least one target antigen is derived from a virus.

6. The method according to claim 5, wherein the virus is HBV or HCV.

7. The method according to claim 1, wherein the prime boost treatment regimen comprises the steps of:
a) administering the polypeptide as a first priming composition; and
b) administering a second boosting composition comprising a vector encoding one or more CTL epitopes of the target antigen, including at least one CTL epitope which is the same as a CTL epitope of the priming composition.

8. The method according to claim 7, wherein the steps (a) and (b) are separated by at least two weeks.

9. The method according to claim 1, wherein the polypeptide is formulated in alum.

10. The method according to claim 7, wherein the vector is a plasmid, a viral vector, a bacterial vector or a yeast vector.

11. The method according to claim 10, wherein the viral vector is a poxvirus vector.

12. The method according to claim 11, wherein the vector is a vaccinia virus vector.

13. The method according to claim 10, wherein the vector is a non-replicating or replication impaired vector.

14. The method according to claim 13, wherein the vector is MVA.

15. The method according to claim 1, wherein the polypeptide is the result of a bacterial or yeast expression.

16. The method according to claim 1, wherein the polyepitope construct is a recombinant string of two or more CTL epitopes.

17. The method according to claim 7, wherein the vector further encodes for one or more HTL epitopes.

18. The method according to claim 17, wherein the HTL epitope is a PADRE® epitope.

19. The method according to claim 1, wherein the two or more CTL epitopes are derived from the HBV Core protein, the HBV polymerase protein and/or the HBV Envelope protein.

20. The method according to claim 19, wherein the two or more CTL epitopes are selected from the list of HBV epitopes given in Table 1.

21. The method according to claim 1, wherein the two or more CTL epitopes are derived from the HCV Core, E1, E2, NS3, NS4 and/or NS5 protein.

22. The method according to claim 21, wherein the two or more CTL epitopes are selected from the list of HCV epitopes given in Table 3.

23. The method according to claim 20, wherein the CTL epitopes include all of the epitopes given in table 1.

24. The method according to claim 22, wherein the CTL epitopes include all of the epitopes given in table 3.

25. The method according to claim 3, wherein the at least one HTL epitope is selected from the list given in Table 2.

26. The method according to claim 3, wherein the at least one HTL epitope is selected from the list given in Table 4.

27. The method according to claim 1, wherein the epitopes of the priming composition are the same as the epitopes of the boosting composition.

28. The method according to claim 1, wherein the priming and/or boosting composition is in particulate form suitable for intravenous, intraepidermal, subcutaneous, intradermal, transdermal, or intramuscular delivery.

29. The method according to claim 1, wherein the T cell response comprises a Cytotoxic T Lymphocyte (CTL) response and optionally a T helper (HTL) response.

30. A kit for inducing a T cell response against at least one target antigen, said kit comprising a priming polypeptide comprising a polyepitope construct comprising two or more CTL epitopes of the target antigen, wherein the polypeptide is not linked to, included within or combined with a compound selected from the group consisting of: a particle formation promoting protein, a carrier protein and a CTL response inducing adjuvant.

31. The kit according to claim 30, further comprising a boosting vector encoding one or more CTL epitopes of the target antigen, including at least one CTL epitope which is the same as a CTL epitope of the priming composition.


SEQUENCE LISTING (106 sequences)

SEQ ID NO: 1 - protein, 9 aa, Hepatitis B Virus
FLLSLGIHL

SEQ ID NO: 2 - protein, 9 aa, Hepatitis B Virus
KYTSFPWLL

SEQ ID NO: 3 - protein, 11 aa, Hepatitis B Virus
RFSWLSLLVPF

SEQ ID NO: 4 - protein, 10 aa, Hepatitis B Virus
FPHCLAFSYM

SEQ ID NO: 5 - protein, 10 aa, Hepatitis B Virus
LVVDFSQFSR

SEQ ID NO: 6 - protein, 10 aa, Hepatitis B Virus
ILLLCLIFLL

SEQ ID NO: 7 - protein, 11 aa, Hepatitis B Virus
HTLWKAGILYK

SEQ ID NO: 8 - protein, 11 aa, Hepatitis B Virus
WMMWYWGPSLY

SEQ ID NO: 9 - protein, 11 aa, Hepatitis B Virus
YPALMPLYACI

SEQ ID NO: 10 - protein, 9 aa, Hepatitis B Virus
WLSLLVPFV

SEQ ID NO: 11 - protein, 9 aa, Hepatitis B Virus
FLLTRILTI

SEQ ID NO: 12 - protein, 9 aa, Hepatitis B Virus
IPIPSSWAF

SEQ ID NO: 13 - protein, 9 aa, Hepatitis B Virus
EYLVSFGVW

SEQ ID NO: 14 - protein, 9 aa, Hepatitis B Virus
LPSDFFPSV

SEQ ID NO: 15 - protein, 10 aa, Hepatitis B Virus
FLPSDFFPSV

SEQ ID NO: 16 - protein, 10 aa, Hepatitis B Virus
DLLDTASALY

SEQ ID NO: 17 - protein, 10 aa, Hepatitis B Virus
SWPKFAVPNL

SEQ ID NO: 18 - protein, 9 aa, Hepatitis B Virus
SAICSVVRR

SEQ ID NO: 19 - protein, 6 aa, Hepatitis B Virus
LSLDVS

SEQ ID NO: 20 - protein, 9 aa, Hepatitis B Virus
NVSIPWTHK

SEQ ID NO: 21 - protein, 9 aa, Hepatitis B Virus
GLSRYVARL

SEQ ID NO: 22 - protein, 11 aa, Hepatitis B Virus
STLPETTVVRR

SEQ ID NO: 23 - protein, 9 aa, Hepatitis B Virus
HPAAMPHLL

SEQ ID NO: 24 - protein, 10 aa, Hepatitis B Virus
RWMCLRRFII

SEQ ID NO: 25 - protein, 8 aa, Hepatitis B Virus
ASFCGSPY

SEQ ID NO: 26 - protein, 9 aa, Hepatitis B Virus
YMDDVVLGV

SEQ ID NO: 27 - protein, 10 aa, Hepatitis B Virus
LWFHISCLTF

SEQ ID NO: 28 - protein, 10 aa, Hepatitis B Virus
TPARVTGGVF

SEQ ID NO: 29 - protein, 11 aa, Hepatitis B Virus
LTFGRETVLEY

SEQ ID NO: 30 - protein, 10 aa, Hepatitis B Virus
QAFTFSPTYK

SEQ ID NO: 31 - protein, 15 aa, Hepatitis B Virus
GTSFVYVPSALNPAD

SEQ ID NO: 32 - protein, 15 aa, Hepatitis B Virus
LCQVFADATPTGWGL

SEQ ID NO: 33 - protein, 15 aa, Hepatitis B Virus
RHYLHTLWKAGILYK

SEQ ID NO: 34 - protein, 20 aa, Hepatitis B Virus
PHHTALRQAILCWGELMTLA

SEQ ID NO: 35 - protein, 15 aa, Hepatitis B Virus
ESRLVVDFSQFSRGN

SEQ ID NO: 36 - protein, 15 aa, Hepatitis B Virus
PFLLAQFTSAICSVV

SEQ ID NO: 37 - protein, 15 aa, Hepatitis B Virus
LVPFVQWFVGLSPTV

SEQ ID NO: 38 - protein, 15 aa, Hepatitis B Virus
LHLYSHPIILGFRKI

SEQ ID NO: 39 - protein, 15 aa, Hepatitis B Virus
SSNLSWLSLDVSAAF

SEQ ID NO: 40 - protein, 15 aa, Hepatitis B Virus
LQSLTNLLSSNLSWL

SEQ ID NO: 41 - protein, 15 aa, Hepatitis B Virus
AGFFLLTRILTIPQS

SEQ ID NO: 42 - protein, 20 aa, Hepatitis B Virus
VSFGVWIRTPPAYRPPNAPI

SEQ ID NO: 43 - protein, 15 aa, Hepatitis B Virus
VGPLTVNEKRRLKLI

SEQ ID NO: 44 - protein, 15 aa, Hepatitis B Virus
KQCFRKLPVNRPIDW

SEQ ID NO: 45 - protein, 15 aa, Hepatitis B Virus
AANWILRGTSFVYVP

SEQ ID NO: 46 - protein, 15 aa, Hepatitis B Virus
KQAFTFSPTYKAFLC

SEQ ID NO: 47 - protein, 13 aa, Hepatitis B Virus
AKFVAAWTLKAAA

SEQ ID NO: 48 - protein, 10 aa, Hepatitis C Virus
VATDALMTGY

SEQ ID NO: 49 - protein, 10 aa, Hepatitis C Virus
FTDNSSPPAV

SEQ ID NO: 50 - protein, 10 aa, Hepatitis C Virus
LVDILAGYGA

SEQ ID NO: 51 - protein, 10 aa, Hepatitis C Virus
VLVGGVLAAL

SEQ ID NO: 52 - protein, 10 aa, Hepatitis C Virus
YLLPRRGPRL

SEQ ID NO: 53 - protein, 9 aa, Hepatitis C Virus
HMWNFISGI

SEQ ID NO: 54 - protein, 9 aa, Hepatitis C Virus
TLWARMILM

SEQ ID NO: 55 - protein, 10 aa, Hepatitis C Virus
GLLGCIITSL

SEQ ID NO: 56 - protein, 9 aa, Hepatitis C Virus
KLQDCTMLV

SEQ ID NO: 57 - protein, 9 aa, Hepatitis C Virus
NIIMYAPTL

SEQ ID NO: 58 - protein, 9 aa, Hepatitis C Virus
AVCTRGVAK

SEQ ID NO: 59 - protein, 9 aa, Hepatitis C Virus
KTSERSQPR

SEQ ID NO: 60 - protein, 9 aa, Hepatitis C Virus
STNPKPQRK

SEQ ID NO: 61 - protein, 9 aa, Hepatitis C Virus
AAYAAQGYK

SEQ ID NO: 62 - protein, 9 aa, Hepatitis C Virus
QYLAGLSTL

SEQ ID NO: 63 - protein, 9 aa, Hepatitis C Virus
MYTNVDQDL

SEQ ID NO: 64 - protein, 10 aa, Hepatitis C Virus
FWAKHMWNFI

SEQ ID NO: 65 - protein, 9 aa, Hepatitis C Virus
VIKGGRHLI

SEQ ID NO: 66 - protein, 9 aa, Hepatitis C Virus
CLIRLKPTL

SEQ ID NO: 67 - protein, 10 aa, Hepatitis C Virus
LPRRGPRLGV

SEQ ID NO: 68 - protein, 10 aa, Hepatitis C Virus
APTLWARMIL

SEQ ID NO: 69 - protein, 10 aa, Hepatitis C Virus
LPINALSNSL

SEQ ID NO: 70 - protein, 9 aa, Hepatitis C Virus
SFSIFLLAL

SEQ ID NO: 71 - protein, 10 aa, Hepatitis C Virus
AYYRGLDVSV

SEQ ID NO: 72 - protein, 10 aa, Hepatitis C Virus
YRRCRASGVL

SEQ ID NO: 73 - protein, 9 aa, Hepatitis C Virus
DPRRRSRNL

SEQ ID NO: 74 - protein, 9 aa, Hepatitis C Virus
GPRLGVRAT

SEQ ID NO: 75 - protein, 9 aa, Hepatitis C Virus
LPGCSFSIF

SEQ ID NO: 76 - protein, 9 aa, Hepatitis C Virus
LIRLKPTLH

SEQ ID NO: 77 - protein, 9 aa, Hepatitis C Virus
HPNIEEVAL

SEQ ID NO: 78 - protein, 9 aa, Hepatitis C Virus
FPYLVAYQA

SEQ ID NO: 79 - protein, 9 aa, Hepatitis C Virus
AETAGARLV

SEQ ID NO: 80 - protein, 9 aa, Hepatitis C Virus
AEQFKQKAL

SEQ ID NO: 81 - protein, 9 aa, Hepatitis C Virus
DYPYRLWHY

SEQ ID NO: 82 - protein, 15 aa, Hepatitis C Virus
AVGIFRAAVCTRGVA

SEQ ID NO: 83 - protein, 14 aa, Hepatitis C Virus
GYKVLVLNPSVAAT

SEQ ID NO: 84 - protein, 14 aa, Hepatitis C Virus
GRHLIFCHSKKKCD

SEQ ID NO: 85 - protein, 19 aa, Hepatitis C Virus
CVTQTVDFSLDPTFTIETT

SEQ ID NO: 86 - protein, 20 aa, Hepatitis C Virus
TPAETTVRLRAYMNTPGLPV

SEQ ID NO: 87 - protein, 16 aa, Hepatitis C Virus
TSTWVLVGGVLAALAA

SEQ ID NO: 88 - protein, 15 aa, Hepatitis C Virus
GKPAIIPDREVLYRE

SEQ ID NO: 89 - protein, 16 aa, Hepatitis C Virus
GIQYLAGLSTLPGNPA

SEQ ID NO: 90 - protein, 15 aa, Hepatitis C Virus
STLLFNILGGWVAAQ

SEQ ID NO: 91 - protein, 18 aa, Hepatitis C Virus
EDLVNLLPAILSPGALVV

SEQ ID NO: 92 - protein, 16 aa, Hepatitis C Virus
GEGAVQWMNRLIAFAS

SEQ ID NO: 93 - protein, 16 aa, Hepatitis C Virus
MNRLIAFASRGNHVSP

SEQ ID NO: 94 - protein, 15 aa, Hepatitis C Virus
SMSYTWTGALITPCA

SEQ ID NO: 95 - protein, 725 aa, Artificial sequence
  HBV Polyepitope Protein
MGTSFVYVPSALNPADGPGPGLCQVFADATPTGWGLGPGPGRHYLHTLWKAGILYKGPGPGPHHTALRQAILCWGELMTLAGPGPGESRLVVDFSQFSRGNGPGPGPFLLAQFTSAICSVVGPGPGLVPFVQWFVGLSPTVGPGPGLHLYSHPIILGFRKIGPGPGSSNLSWLSLDVSAAFGPGPGLQSLTNLLSSNLSWLGPGPGAGFFLLTRILTIPQSGPGPGVSFGVWIRTPPAYRPPNAPIGPGPGVGPLTVNEKRRLKLIGPGPGKQCFRKLPVNRPIDWGPGPGAANWILRGTSFVYVPGPGPGKQAFTFSPTYKAFLCGPGPGFLLSLGIHLNAAAKYTSFPWLLNAAARFSWLSLLVPFNAAFPHCLAFSYMKAALVVDFSQFSRGAILLLCLIFLLNAAAHTLWKAGILYKKAWMMWYWGPSLYKAYPALMPLYACIGAAAWLSLLVPFVNAAAGFLLTRILTINAAAIPIPSSWAFKAAAEYLVSFGVWNLPSDFFPSVKAAAFLPSDFFPSVKAAADLLDTASALYNSWPKFAVPNLKAAASAICSVVRRKLSLDVSAAFYNAAAKFVAAWTLKAAAKAANVSIPWTHKGAAGLSRYVARLNAAASTLPETTVVRRKHPAAMPHLLKAAARWMCLRRFIINASFCGSPYKAAYMDDVVLGVNALWFHISCLTFKAAATPARVTGGVFKAAALTFGRETVLEYKQAFTFSPTYK

SEQ ID NO: 96 - protein, 665 aa, Artificial sequence
  HCV Polyepitope Protein
MGRHLIFCHSKKKCDGPGPGSTLLFNILGGWVAAQGPGPGCVTQTVDFSLDPTFTIETTGPGPGEDLVNLLPAILSPGALVVGPGPGGEGAVQWMNRLIAFASGPGPGMNRLIAFASRGNHVSPGPGPGSMSYTWTGALITPCAGPGPGTPAETTVRLRAYMNTPGLPVGPGPGAVGIFRAAVCTRGVAGPGPGGIQYLAGLSTLPGNPAGPGPGTSTWVLVGGVLAALAAGPGPGGYKVLVLNPSVAATGPGPGGKPAIIPDREVLYREKAVIKGGRHLIKAGPRLGVRATKAAAQYLAGLSTLNAAAPTLWARMILNAAHPNIEEVALNLVDILAGYGAKHMWNFISGINAYYRGLDVSVKLQDCTMLVNAAAAEQFKQKALKTSERSQPRNAAFPYLVAYQAKAAMYTNVDQDLNTLWARMILMNLPINALSNSLKSTNPKPQRKNDYPYRLWHYKAACLIRLKPTLNIIMYAPTLKAAVATDALMTGYNLPGCSFSIFKYRRCRASGVLKAAVLVGGVLAALNGLLGCIITSLNAAYAAQGYKKDPRRRSRNLKAAAYLLPRRGPRLNFWAKHMWNFIKAAAKFVAAWTLKAAAKALIRLKPTLHKAAAVCTRGVAKNFTDNSSPPAVKALPRRGPRLGVKSFSIFLLALKAAETAGARLV

SEQ ID NO: 97 - protein, 17 aa, Artificial sequence
  Tag
HHMFHHHWWHHHMWHHH

SEQ ID NO: 98 - DNA, 2217 bp, Artificial sequence
  HBV Polyepitope construct with linker and tag His6
atgggcacaagctttgtgtatgtcccgtcggccttaaacccagcggatggtccggggcctggcctctgccaggtgtttgcagatgccacccctaccgggtggggtctgggcccgggtccgggtcgtcattacctgcatacgttgtggaaagctggcatcctttataaaggcccaggtcctggtccacatcacactgcgttacgtcaggctatcctttgctggggcgaactgatgacgctggcggggcctggcccgggtgaaagtcgcctggttgtagatttctcccaatttagccgtgggaatggtccgggcccgggtccgtttttactggcacagttcacatcagcgatctgctcggtcgttgggccaggtcctggcctggtaccttttgttcagtggtttgttggtcttagtccgaccgtcggtccggggccaggtcttcacctgtactcccacccgatcattttaggttttcgcaaaatcggccctggcccaggctctagcaacttatcctggctgtcactcgacgtgagcgcggctttcggcccaggtccagggctccagtctctcaccaacctgttgagttctaatctgtcttggttgggcccgggtcctggcgctggcttctttctgcttacacgcattctgacgatcccgcaatcagggccaggcccgggtgtgagcttcggggtttggattcgtaccccgccagcctatcgtccaccgaacgcaccgattggcccaggtccgggggtgggtcctctgactgtaaatgagaaacgtcgcttgaaattgattggtccaggcccgggcaaacaatgtttccgtaaactgcctgtgaaccgtccgattgattggggcccggggccgggtgcagcgaattggattctgcgcgggacctcgtttgtgtatgtccctggcccgggtccgggcaagcaagcgtttacgttcagtccgacatacaaggccttcctgtgtggtcctggtccaggtttcctgctaagcttgggtatccacctgaacgccgcagcgaagtacacgagctttccgtggttacttaatgcggccgctcgcttctcttggctgtcccttctggtgccgtttaacgcagctttcccacactgcctggcgttttcctacatgaaggcagcgctggtagtggatttttcacaattctcccgcggcgcgattctgttactgtgccttatcttcctgttgaacgctgccgcacataccctttggaaagccgggattttgtataaaaaggcatggatgatgtggtactggggcccgagtttatacaaagcctatcctgctctcatgccgttgtatgcatgtatcggcgctgcggcttggctgtcactcctggtgccatttgtaaacgcagccgctggcttcctgttgacacgcattctgaccatcaatgcagctgcaattccgatcccgtcgagttgggcgtttaaggcggcagccgaatatctggtgtcatttggtgtgtggaacttaccgagcgactttttcccatctgttaaagcggccgcgttcctcccaagtgattttttcccgagtgtcaaggccgctgccgatctgctcgatacagcctcggcgctgtataattcttggcctaaatttgcggttcctaatctgaaagccgcggctagtgccatttgcagcgttgtccgtcgcaaattatcactcgacgtgagcgcagccttttataacgccgcggccaaatttgtggcggcctggacgctgaaagcagcggctaaagcggctaatgtttcgattccgtggactcataaaggtgcagcgggcctgtctcgctatgttgcgcgtcttaacgccgcagcctcgactcttcctgagactacggtggtccgtcgtaaacatccggcggccatgccacatctgttaaaagcggcagcgcgttggatgtgtttgcgccgttttattatcaatgcgagcttttgtgggagcccgtacaaagcggcatacatggacgatgttgtcttaggggtaaatgcgctgtggttccacatttcttgcctgaccttcaaagccgctgcgaccccggcacgtgtcaccggtggcgtattcaaagcggctgcgttaacctttggtcgtgaaacggttctggaatataagcaggcatttacattttcccctacctataaaaacgcgccgggatccctcgagcaccaccaccaccaccactaa

SEQ ID NO: 99 - DNA, 2238 bp, Artificial sequence
  HBV polyepitope construct with linker and tag LHH-11
atgggcacaagctttgtgtatgtcccgtcggccttaaacccagcggatggtccggggcctggcctctgccaggtgtttgcagatgccacccctaccgggtggggtctgggcccgggtccgggtcgtcattacctgcatacgttgtggaaagctggcatcctttataaaggcccaggtcctggtccacatcacactgcgttacgtcaggctatcctttgctggggcgaactgatgacgctggcggggcctggcccgggtgaaagtcgcctggttgtagatttctcccaatttagccgtgggaatggtccgggcccgggtccgtttttactggcacagttcacatcagcgatctgctcggtcgttgggccaggtcctggcctggtaccttttgttcagtggtttgttggtcttagtccgaccgtcggtccggggccaggtcttcacctgtactcccacccgatcattttaggttttcgcaaaatcggccctggcccaggctctagcaacttatcctggctgtcactcgacgtgagcgcggctttcggcccaggtccagggctccagtctctcaccaacctgttgagttctaatctgtcttggttgggcccgggtcctggcgctggcttctttctgcttacacgcattctgacgatcccgcaatcagggccaggcccgggtgtgagcttcggggtttggattcgtaccccgccagcctatcgtccaccgaacgcaccgattggcccaggtccgggggtgggtcctctgactgtaaatgagaaacgtcgcttgaaattgattggtccaggcccgggcaaacaatgtttccgtaaactgcctgtgaaccgtccgattgattggggcccggggccgggtgcagcgaattggattctgcgcgggacctcgtttgtgtatgtccctggcccgggtccgggcaagcaagcgtttacgttcagtccgacatacaaggccttcctgtgtggtcctggtccaggtttcctgctaagcttgggtatccacctgaacgccgcagcgaagtacacgagctttccgtggttacttaatgcggccgctcgcttctcttggctgtcccttctggtgccgtttaacgcagctttcccacactgcctggcgttttcctacatgaaggcagcgctggtagtggatttttcacaattctcccgcggcgcgattctgttactgtgccttatcttcctgttgaacgctgccgcacataccctttggaaagccgggattttgtataaaaaggcatggatgatgtggtactggggcccgagtttatacaaagcctatcctgctctcatgccgttgtatgcatgtatcggcgctgcggcttggctgtcactcctggtgccatttgtaaacgcagccgctggcttcctgttgacacgcattctgaccatcaatgcagctgcaattccgatcccgtcgagttgggcgtttaaggcggcagccgaatatctggtgtcatttggtgtgtggaacttaccgagcgactttttcccatctgttaaagcggccgcgttcctcccaagtgattttttcccgagtgtcaaggccgctgccgatctgctcgatacagcctcggcgctgtataattcttggcctaaatttgcggttcctaatctgaaagccgcggctagtgccatttgcagcgttgtccgtcgcaaattatcactcgacgtgagcgcagccttttataacgccgcggccaaatttgtggcggcctggacgctgaaagcagcggctaaagcggctaatgtttcgattccgtggactcataaaggtgcagcgggcctgtctcgctatgttgcgcgtcttaacgccgcagcctcgactcttcctgagactacggtggtccgtcgtaaacatccggcggccatgccacatctgttaaaagcggcagcgcgttggatgtgtttgcgccgttttattatcaatgcgagcttttgtgggagcccgtacaaagcggcatacatggacgatgttgtcttaggggtaaatgcgctgtggttccacatttcttgcctgaccttcaaagccgctgcgaccccggcacgtgtcaccggtggcgtattcaaagcggctgcgttaacctttggtcgtgaaacggttctggaatataagcaggcatttacattttcccctacctataaaaacgcgcatcaccatatgtttcaccatcattggtggcatcatcacatgtggcaccatcactaa

SEQ ID NO: 100 - DNA, 2061 bp, Artificial sequence
  HCV polyepitope construct with linker and tag LHH-11
atgggccgtcatctgattttttgccacagcaaaaaaaaatgcgatgggccgggtcctggtagcaccctgctgtttaacattctgggcggctgggttgcggcgcagggtccaggtccgggctgtgttacccagaccgtggattttagcctggacccgacctttaccattgaaaccactgggccaggtcctggggaggacctggtgaacctgctgccggcgattctgtctccgggtgcgctggttgttggtccaggtccggggggtgaaggtgcggtgcagtggatgaaccgtctgattgcgtttgcgtctggtccagggccaggtatgaaccgcctgatcgcgttcgccagccgtggcaaccatgtttctccgggtccagggccaggtagcatgagctatacctggaccggtgcgctgattaccccgtgtgcaggtccagggccgggtacgccggcggaaaccaccgttcgtctgcgtgcgtatatgaacacgccgggtctgccggtgggtccaggtccaggtgcggtgggcatttttcgtgcggcggtgtgcacccgtggtgttgcaggcccaggtccagggggtattcagtatctggccggcctgagcaccctgccgggtaatccggcgggtccgggtccaggcacctctacctgggtgctggttggtggtgttctggccgcgctggccgcaggtccgggtccagggggctataaagtgctggtgctgaatccgagcgttgcggcgaccggtccgggtccgggtggtaaaccggcgattattccggatcgtgaagtgctgtatcgtgaaaaagcggtgattaaaggcggccgtcatctgattaaagcgggtccacgcctgggtgttcgtgcgaccaaagccgcagcgcagtatctggccggtctgagcaccctgaatgcggcagcgccgacgctgtgggcgcgtatgattctgaacgcggcgcatccgaacattgaagaagtggcgctgaacctggtggatattctggccggctatggcgcaaaacatatgtggaactttatcagcggcatcaacgcgtattatcgtggcctggatgtgagcgtgaaactgcaggattgcaccatgctggtgaatgcggccgcagcggaacagtttaaacagaaagcgctgaaaaccagcgaacgtagccagccgcgtaacgcggcgtttccgtatctggtggcgtatcaggcgaaagcggcgatgtataccaacgtggatcaggatctgaacaccctgtgggcccgcatgatcctgatgaacctgccgattaacgccctgagcaacagcctgaaaagcaccaatccgaaaccgcagcgtaaaaacgattatccgtatcgcctgtggcattataaagcggcgtgcctgattcgtctgaaaccaactctgaacattattatgtatgccccgacgctgaaagcagccgttgcgaccgatgcgctgatgaccggctataacctgccgggctgcagctttagcatttttaaatatcgtcgttgccgtgcgagcggcgttctgaaagctgcggtgctggttggtggtgttctggccgcgctgaacggtctgctgggctgcattattaccagcctgaacgcggcctatgcggcgcagggctataaaaaagatccgcgtcgtcgtagccgtaacctgaaagcagcggcgtatctgctgccgcgccgtggcccgcgtctgaacttttgggcgaaacacatgtggaatttcattaaagccgcggccaaatttgtggcggcgtggaccctgaaagccgccgcgaaagcgctgatccgcctgaaaccgaccctgcataaagcggcagcggtgtgcacccgtggcgtggcgaaaaactttaccgataacagctctccgccggcagttaaagccctgccgcgtcgcggtccgcgcctgggcgtgaaatcttttagcatctttctgctggccctgaaagcggcggaaaccgcgggtgcgcgtctggttaatgcggcgcatcatcatatgtttcaccatcattggtggcatcatcacatgtggcaccatcactaa

SEQ ID NO: 101 - DNA, 4947 bp, Artificial sequence
  Plasmid pAcI
gttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcaggtgatgattatcagccagcagagaattaaggaaaacagacaggtttattgagcgcttatctttccctttatttttgctgcggtaagtcgcataaaaaccattcttcataattcaatccatttactatgttatgttctgaggggagtgaaaattcccctaattcgatgaagattcttgctcaattgttatcagctatgcgccgaccagaacaccttgccgatcagccaaacgtctcttcaggccactgactagcgataactttccccacaacggaacaactctcattgcatgggatcattgggtactgtgggtttagtggttgtaaaaacacctgaccgctatccctgatcagtttcttgaaggtaaactcatcacccccaagtctggctatgcagaaatcacctggctcaacagcctgctcagggtcaacgagaattaacattccgtcaggaaagcttggcttggagcctgttggtgcggtcatggaattaccttcaacctcaagccagaatgcagaatcactggcttttttggttgtgcttacccatctctccgcatcacctttggtaaaggttctaagcttaggtgagaacatccctgcctgaacatgagaaaaaacagggtactcatactcacttctaagtgacggctgcatactaaccgcttcatacatctcgtagatttctctggcgattgaagggctaaattcttcaacgctaactttgagaatttttgtaagcaatgcggcgttataagcatttaatgcattgatgccattaaataaagcaccaacgcctgactgccccatccccatcttgtctgcgacagattcctgggataagccaagttcatttttctttttttcataaattgctttaaggcgacgtgcgtcctcaagctgctcttgtgttaatggtttcttttttgtgctcatacgttaaatctatcaccgcaagggataaatatctaacaccgtgcgtgttgtaccgagctcgaattgctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctcaaagatgcaggggtaaaagctaaccgcatctttaccgacaaggcatccggcagttcaacagatcgggaagggctggatttgctgaggatgaaggtggaggaaggtgatgtcattctggtgaagaagctcgaccgtcttggccgcgacaccgccgacatgatccaactgataaaagagtttgatgctcagggtgtagcggttcggtttattgacgacgggatcagtaccgacggtgatatggggcaaatggtggtcaccatcctgtcggctgtggcacaggctgaacgccggaggatcctagagcgcacgaatgagggccgacaggaagcaaagctgaaaggaatcaaatttggccgcaggcgtaccgtggacaggaacgtcgtgctgacgcttcatcagaagggcactggtgcaacggaaattgctcatcagctcagtattgcccgctccacggtttataaaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgt

SEQ ID NO: 102 - DNA, 4182 bp, Artificial sequence
  Plasmid pcI857
tctcgatgatggttacgccagactatcaaatatgctgcttgaggcttattcgggcgcagatctttagctgtcttggtttgcccaaagcgcattgcataatctttcagggttatgcgttgttccatacaacctccttagtacatgcaaccattatcaccgccagaggtaaaatagtcaacacgcacggtgttagatatttatcccttgcggtgatagatttaacgtatgagcacaaaaaagaaaccattaacacaagagcagcttgaggacgcacgtcgccttaaagcaatttatgaaaaaaagaaaaatgaacttggcttatcccaggaatctgtcgcagacaagatggggatggggcagtcaggcgttggtgctttatttaatggcatcaatgcattaaatgcttataacgccgcattgcttacaaaaattctcaaagttagcgttgaagaatttagcccttcaatcgccagagaaatctacgagatgtatgaagcggttagtatgcagccgtcacttagaagtgagtatgagtaccctgttttttctcatgttcaggcagggatgttctcacctaagcttagaacctttaccaaaggtgatgcggagagatgggtaagcacaaccaaaaaagccagtgattctgcattctggcttgaggttgaaggtaattccatgaccgcaccaacaggctccaagccaagctttcctgacggaatgttaattctcgttgaccctgagcaggctgttgagccaggtgatttctgcatagccagacttgggggtgatgagtttaccttcaagaaactgatcagggatagcggtcaggtgtttttacaaccactaaacccacagtacccaatgatcccatgcaatgagagttgttccgttgtggggaaagttatcgctagtcagtggcctgaagagacgtttggctgatcggcaaggtgttctggtcggcgcatagctgataacaattgagcaagaatcttcatcgaattaggggaattttcactcccctcagaacataacatagtaaatggattgaattatgaagaatggtttttatgcgacttaccgcagcaaaaataaagggaaagataagcgctcaataaacctgtctgttttccttaattctctgctggctgataatcatcacctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctcaaagatgcaggggtaaaagctaaccgcatctttaccgacaaggcatccggcagttcaacagatcgggaagggctggatttgctgaggatgaaggtggaggaaggtgatgtcattctggtgaagaagctcgaccgtcttggccgcgacacgccgacatgatccaactgataaaagagtttgatgctcagggtgtagcggttcggtttattgacgacgggatcagtaccgacggtgatatggggcaaatggtggtcaccatcctgtcggctgtggcacaggctgaacgccggagga

SEQ ID NO: 103 - protein, 5 aa, Artificial sequence
  Spacer sequence
GPGPG

SEQ ID NO: 104 - protein, 22 aa, Artificial sequence
  IGK Signal sequence
MGMQVQIQSLFLLLLWVPGSRG

SEQ ID NO: 105 - protein, 6 aa, Artificial sequence
  His6 tag
HHHHHH

SEQ ID NO: 106 - protein, 5 aa, Artificial Sequence
  linker sequence
PGSLE